(19) United States
(12) Patent Application Publication
(10) Pub. No.: US 2019/0351024 A1
(43) Pub. Date: Nov. 21, 2019

(54) IMMUNOCYTOKINES WITH PROGRESSIVE ACTIVATION MECHANISM

(71) Applicant: PHILOGEN S.p.A., Siena (IT)

(72) Inventors: Giovanni Neri, Siena (IT); Alessandra Villa, Zurich (CH); Florent Samain, Regensdorf (CH); Martina Bigatti, Zurich (CH)

(21) Appl. No.: 16/471,509

(22) PCT Filed: Dec. 21, 2017
(86) PCT No.: PCT/EP2017/084256

§ 371 Date: Jun. 19, 2019

(30) Foreign Application Priority Data
Dec. 21, 2016 (GB) 1621806.7

(51) Int. Cl. A61K 38/20; A61K 39/395; A61K 31/4045; A61P 35/00

(52) U.S. Cl. CPC A61K 38/2013; A61K 39/39558; A61K 2039/55533; A61P 35/00; A61K 38/208; A61K 31/4045

(57) ABSTRACT

The present invention relates to a combination comprising at least an immunocytokine comprising at least a primary binding protein or peptide and a cytokine, fused or conjugated to one another, and a secondary binding molecule capable of binding to at least a section of at least one cytokine comprised in the immunocytokine.

### FIELD OF THE INVENTION

The present invention relates to the field of immunocytokines.

### INTRODUCTION

A number of recombinant cytokine products are routinely used in the clinic (e.g., IL2, interferon-α, interferon-α, interferon-γ, G-CSF, GM-CSF, TNF) for various therapeutic indication. Many other cytokines have been investigated in clinical trials.

While many cytokine products provide a clinical benefit to patients, they can often be toxic at low doses. This applies, especially, for pro-inflammatory cytokines, and may therefore restrict the therapeutic window of these drugs, hence restricting dose escalation to therapeutically effective regimens.

In order to improve the therapeutic index—i.e., the ratio between the amount of the cytokine that causes the therapeutic effect to the amount that causes toxicity, a cytokine can be conveniently fused or conjugated to a suitable binding protein or peptide, preferably an antibody, a modified antibody format, an antibody derivative or fragment retaining target binding properties, an antibody-based binding protein, a peptide binder and/or an antibody mimetic, which then serves as a pharmacodelivery vehicle. These constructs are often called “immunocytokines”.

In preclinical models of cancer (Pasche and Neri, 2012, Drug Discov Today, 17, 583-590) and of chronic inflammation (Bootz and Neri, 2016, Drug Discov Today, 21, 180-189), the superiority of certain immunocytokines compared to the corresponding unmodified cytokines has been demonstrated.

In addition, encouraging results are emerging from clinical trials, using immunocytokine products in patients (Danielli et al., 2015, Cancer Immunol Immunother 64, 999-1009, Papadia et al., 2013, J Surg Oncol, 107, 173-179, Schliemann et al., 2015, Cancer Immunol. Res., 3, 547-556, Gutbrodt et al. (2013) Sci. Trans. Med., 5, 201ra118).

While immunocytokines exhibit promising therapeutic results, these products are sometimes associated with side effects including toxicities. In many cases, the antibody-cytokine fusion protein has an in vitro activity similar to the naked cytokine. Consequently, the in vivo tolerability profile is often similar at equal doses, even though the targeted immunocytokine product is typically superior to the non-targeted counterpart in terms of activity.

This issue has been addressed by a number of strategies disclosed in the art:

Venetz et al. (2016, J. Biol. Chem, 291, 18139-18147) have shown that the targeted assembly of antibody products upon antigen binding may represent a novel strategy for the reconstitution of potent therapeutic activity at the site of disease, avoiding healthy tissues. The authors demonstrated that interleukin-12 (IL12), a heterodimeric pro-inflammatory cytokine consisting of the disulfide-linked p40 and p35 subunits, can be reconstituted by sequential reassembly of fusion proteins based on antibody fragments and interleukin-12 subunit mutants. In principle, this strategy should facilitate the development of an inflammatory response at the site of disease, minimizing systemic toxicity. However this principle can only be applied for multimeric cytokines, like IL12, IL23, IL24, IL10, IL35, and is troublesome when it comes to translation into the clinic (regulatory issues, correct dosage, patient compliance, manufacturing issues, and the like).

Desnoyer et al. 2016, Sci Transl Med, 5, 207ra144 suggest to use inactive cytokine precursors as immunocytokine payloads. These could be activated at the site of disease by tissue- or disease specific proteolytic processing. This strategy has been successfully implemented for monoclonal antibodies, leading to an improved therapeutic index in mice. However, the scope of this approach is restricted to target tissues or tumors exhibiting increased proteolytic activity. Further, this approach likewise raises questions as regards regulatory issues and correct dosage.

Other authors have suggested the use of antibody-cytokine complexes (Boyman and Sprent, 2012. Nat Rev Immunol, 12, 180-190). This strategy aims at achieving the extension of the circulatory half-life of the cytokine product and, simultaneously, at stably conferring novel selectivity profiles, altering the interaction with certain cytokine receptor subunits.

In WO2012/107417, the masking of specific cytokine epitopes, for example in order to bias reactivity of IL2 towards CD8+ T cells or regulatory T cells in the case of IL2, has been achieved by the insertion of amino acid substitutions at crucial residue positions on the cytokine surface. Such approach can not be applied universally, but depends on the possibility to introduce such mutations without impairing cytokine activity.

Another recently described approach employs antibody-IL2 fusion proteins, in which the biological activity of the cytokine moiety is modulated once the antibody binds to its target. This “allosteric” modulation can be explained by the hinge movement of the Fab arms of the antibody upon antigen engagement, and by strategic positioning of the IL2 moiety at the C-terminal end of the light chain and does hence not provide an approach that can be universally applied.

It is hence one object of the present invention to provide an alternative approach to improve the therapeutic index, or increase the therapeutic window, of one or more immunocytokines.

It is another object of the present invention to provide an approach to improve the therapeutic index, or increase the therapeutic window, of one or more immunocytokines, which can be applied universally, i.e., independent of the specific type of cytokine, antibody, target tissue or disease.

It is another object of the present invention to provide an immunocytokine-related approach which reduces side effects caused by said therapy.

### SUMMARY OF THE INVENTION

These and further objects are met with methods and means according to the independent claims of the present invention. The dependent claims are related to specific embodiments.

### EMBODIMENTS OF THE INVENTION

Before the invention is described in detail, it is to be understood that this invention is not limited to the particular component parts or structural features of the devices or compositions described or process steps of the methods described as such devices and methods may vary. It is also to be understood that the terminology used herein is for purposes of describing particular embodiments only, and is not intended to be limiting. The mere fact that certain measures are recited in mutually different dependent claims does not indicate that a combination of these measures cannot be used to advantage. Any reference signs in the claims should not be construed as limiting the scope. It must be noted that, as used in the specification and the appended claims, the singular forms “a,” “an” and “the” include singular and/or plural referents unless the context clearly dictates otherwise. Further, in the claims, the word “comprising” does not exclude other elements or steps. The mere fact that certain measures are recited in mutually different dependent claims does not indicate that a combination of these measures cannot be used to advantage.

It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

According to one embodiment of the invention, a combination is provided comprising
a) an immunocytokine comprising at least
   a1) a primary binding protein or peptide and
   a2) a cytokine
   fused or conjugated to one another, and
b) a secondary binding molecule capable of binding to at least a section of at least one cytokine comprised in the immunocytokine.

In one embodiment of said invention, the primary binding protein or peptide comprises at least one of the group selected from
an antibody,
a modified antibody format,
an antibody derivative or fragment retaining target binding properties
an antibody-based binding protein,
a peptide binder and/or
an antibody mimetic.

“Antibodies”, also synonymously called “immunoglobulins” (Ig), are generally comprising four polypeptide chains, two heavy (H) chains and two light (L) chains, and are therefore multimeric proteins, or an equivalent Ig homologue thereof (e.g., a camelid nanobody, which comprises only a heavy chain, single domain antibodies (dAbs) which can be either be derived from a heavy or light chain); including full length functional mutants, variants, or derivatives thereof (including, but not limited to, murine, chimeric, humanized and fully human antibodies, which retain the essential epitope binding features of an Ig molecule, and including dual specific, bispecific, multispecific, and dual variable domain immunoglobulins; Immunoglobulin molecules can be of any class (e.g., IgG, IgE, IgM, IgD, IgA, and IgY), or subclass (e.g., IgG1, IgG2, IgG3, IgG4, IgA1, and IgA2) and allotype.

The term “modified antibody format”, as used herein, encompasses antibody-drug-conjugates, Polyalkylene oxide-modified scFv, Monobodies, Diabodies, Camelid Antibodies, Domain Antibodies, bi- or trispecific antibodies, IgA, or two IgG structures joined by a J chain and a secretory component, shark antibodies, new world primate framework+non-new world primate CDR, IgG4 antibodies with hinge region removed, IgG with two additional binding sites engineered into the CH3 domains, antibodies with altered Fc region to enhance affinity for Fc gamma receptors, dimerised constructs comprising CH3+VL+VH, and the like.

An “antibody derivative or fragment”, as used herein, relates to a molecule comprising at least one polypeptide chain derived from an antibody that is not full length, including, but not limited to (i) a Fab fragment, which is a monovalent fragment consisting of the variable light (V_{L}), variable heavy (V_{H}), constant light (C_{L}) and constant heavy 1 (C_{H}1) domains; (ii) a F(ab′)2 fragment, which is a bivalent fragment comprising two Fab fragments linked by a disulfide bridge at the hinge region; (iii) a heavy chain portion of a F_{ab} (F_{d}) fragment, which consists of the V_{H} and C_{H}1 domains; (iv) a variable fragment (Fᵥ) fragment, which consists of the V_{L} and V_{H} domains of a single arm of an antibody, (v) a domain antibody (dAb) fragment, which comprises a single variable domain; (vi) an isolated complementarity determining region (CDR); (vii) a single chain Fᵥ Fragment (scFᵥ); (viii) a diabody, which is a bivalent, bispecific antibody in which V_{H} and V_{L} domains are expressed on a single polypeptide chain, but using a linker that is too short to allow for pairing between the two domains on the same chain, thereby forcing the domains to pair with the complementarity domains of another chain and creating two antigen binding sites; and (ix) a linear antibody, which comprises a pair of tandem Fᵥ segments (V_{H}-C_{H}1-V_{H}-C_{H}1) which, together with complementarity light chain polypeptides, form a pair of antigen binding regions; and (x) other non-full length portions of immunoglobulin heavy and/or light chains, or mutants, variants, or derivatives thereof, alone or in any combination. In any case, said derivative or fragment retains target binding properties

An “antibody-based binding protein”, as used herein, may represent any protein that contains at least one antibody-derived V_{H}, V_{L}, or C_{H} immunoglobulin domain in the context of other non-immunoglobulin, or non-antibody derived components. Such antibody-based proteins include, but are not limited to (i) F_{c}-fusion proteins of binding proteins, including receptors or receptor components with all or parts of the immunoglobulin C_{H} domains, (ii) binding proteins, in which V_{H} and or V_{L} domains are coupled to alternative molecular scaffolds, or (iii) molecules, in which immunoglobulin V_{H}, and/or V_{L}, and/or C_{H} domains are combined and/or assembled in a fashion not normally found in naturally occurring antibodies or antibody fragments.

It is important to understand that the above embodiments, which all rely on the IgG-based antibody concept, can be or rely on any one selected from the list consisting of:

a) hybridoma-derived antibody

b) chimerised antibody

c) humanised antibody, and/or

d) human antibody.

The hybridoma technique is well known in the art, and described by Köhler G and Milstein C, Nature. Bd. 256, 1975, S. 495-497. Methods for the production and/or selection of chimeric, humanised and/or human mAbs are known in the art. For example, U.S. Pat. No. 6,331,415 by Genentech describes the production of chimeric antibodies, while U.S. Pat. No. 6,548,640 by Medical Research Council describes CDR grafting techniques and U.S. Pat. No. 5,859,205 by Celltech describes the production of humanised antibodies.

The term “peptide binder”, as used herein, refers to oligo- or polypeptide, preferably with a length of ≤50 amino acid residues, which have a binding affinity to a given cellular or molecular target. Making or finding suitable peptide binders is described in the art, e.g., in Wada A, Front Immunol. 2013; 4: 224.

The term “antibody mimetic”, as used herein, refers to proteins not belonging to the immunoglobulin family, and even non-proteins such as aptamers, or synthetic polymers. Some types have an antibody-like beta-sheet structure. Potential advantages of “antibody mimetics” or “alternative scaffolds” over antibodies are better solubility, higher tissue penetration, higher stability towards heat and enzymes, and comparatively low production costs. Some antibody mimetics can be provided in large libraries, which offer specific binding candidates against every conceivable target. Just like with antibodies, target specific antibody mimetics can be developed by use of High Throughput Screening (HTS) technologies as well as with established display technologies, just like phage display, bacterial display, yeast or mammalian display. Currently developed antibody mimetics encompass, for example, ankyrin repeat proteins (called DARPins), C-type lectins, A-domain proteins of S. aureus, transferrins, lipocalins, 10th type III domains of fibronectin, Kunitz domain protease inhibitors, ubiquitin derived binders (called affilins), gamma crystallin derived binders, cysteine knots or knottins, thioredoxin A scaffold based binders, SH-3 domains, stradobodies, “A domains” of membrane receptors stabilised by disulfide bonds and Ca2+, CTLA4-based compounds, Fyn SH3, and aptamers (peptide molecules that bind to a specific target molecules). An overview is given in Gebauer M, Skerra A (2009), Curr Opin Chem Biol. 13 (3): 245-255.

Furthermore, routine methods exist to create antibody libraries, peptide libraries or antibody mimetic libraries, to then select, from such libraries, suitable binders against almost all conceivable cellular or molecular targets. In vitro antibody libraries are, among others, disclosed in U.S. Pat. No. 6,300,064 by MorphoSys and U.S. Pat. No. 6,248,516 by MRC/Scripps/Stratagene. Phage Display techniques are for example disclosed in U.S. Pat. No. 5,223,409 by Dyax. Transgenic mammal platforms are for example described in Lonberg N, Nat Biotechnol. 2005 September; 23(9):1117-25.

The “primary binding protein or peptide”, as used herein, can also be called a “targeting protein or peptide”, as it is meant to guide the cytokine to the site of disease, where it binds a disease specific target structure.

The “secondary binding molecule”, as used herein, is also called a “masking molecule”, as it is meant to mask the cytokine and hence inhibits its activity as long as the immunocytokine is travelling through the patient's body to the site of action

In some cases, such secondary binding molecule may only partially reduce the activity of the respective cytokine, e.g., by blocking the interaction with a specific subunit of a given cytokine receptor only. One example of such embodiment would be an antibody, or an antibody derivative or fragment, or a small molecule, which binds to IL2, but which only interferes with the binding thereof to the alpha subunit of the IL2 receptor. Another example of such embodiment would be an antibody, or an antibody derivative or fragment, or a small molecule, which binds to IL12, but which only interferes with the binding thereof to the IL12-R subunit β2. Further examples encompass an antibody, or an antibody derivative or fragment, or a small molecule, that binds to TNFα, and interferes with the binding thereof to TNF-R.

The inventors have realized that a given immunocytokine will progressively accumulate at the site of disease, following injection into a patient (Pasche and Neri, 2012, Drug Discov Today, 17, 583-590).

By masking the cytokine part of a given immunocytokine with a secondary binding molecule, the latter will gradually dissociate from the cytokine part after injection—ideally after the immunocytokine has reached its target and bound thereto, hence allowing the product to progressively gain therapeutic activity and loose toxicity at the same time.

The secondary binding molecule will gradually loose its inhibiting activity over time, because it will be excreted from circulation while the level of the T-cells expressing the cytokine receptor remains constant.

According to another embodiment of said invention, the secondary binding molecule is selected from the group consisting of
a binding protein or peptide
an aptamer, and/or
a small molecule

The term “aptamer” as used herein refers to single-stranded nucleic acid molecules with secondary structures that facilitate high-affinity binding to a target molecule. Aptamers can be synthesized and screened by any suitable methods in the art. For example, aptamers can be screened and identified from a random aptamer library by SELEX (systematic evolution of ligands by exponential enrichment). In such way, aptamers against all conceivable cellular or molecular targets can be found, even if the identity of the molecule is unknown (Phillips et al., 2008, Anal Chim Acta 621:101-108).

The term “small molecule”, as used herein, refers to a non-peptidic, non-oligomeric organic compound either synthesized in the laboratory or found in nature. Small molecules, as used herein, can refer to compounds that are “natural product-like”, however, the term “small molecule” is not limited to “natural product-like” compounds. Rather, a small molecule is typically characterized in that it contains several carbon-carbon bonds, and has preferably a molecular weight of less than 2500 Daltons, although this characterization is not intended to be limiting for the purposes of the present invention. Small molecular libraries can also be created and used for screening for suitable binders against a given target (Dandapani S et al., Curr Protoc Chem Biol. 2012; 4: 177-191).

For the inhibition of IL2 activity, these small molecules are preferably selected from the group of methylindoles, as disclosed in Leimbacher M et al. Chemistry. 2012 Jun. 18; 18(25):7729-37 and in affinity-optimized methylindole variants.

According to another embodiment of the invention, the secondary binding protein or peptide comprises at least one of the group selected from
an antibody,
a modified antibody format,
an antibody derivative or fragment retaining target binding properties
an antibody-based binding protein,
a globular protein,
a peptide binder and/or
an antibody mimetic.

As regards these terms, the definitions set forth above apply. A globular protein, as used herein, can be a protein in a folded structure and can be relatively spherical in shape. Globular proteins include proteins that are more or less soluble in aqueous solutions. There may be a single chain or two or more chains folded together. Portions of the chains may have helical structures, pleated structures, or completely random structures. In some embodiments, a globular protein can be an enzyme. A globular protein generally has a larger molecular weight than a simple peptide or a polypeptide. In some embodiments, the molecular weight of a globular protein is greater than 10 kDa. In some embodiments, the molecular weight of a globular protein is greater than 20 kDa, 30 kDa, or 50 kDa. In some embodiments, the molecular weight of a globular protein may be greater than 100 kDa. In some embodiments, the molecular weight of a globular protein ranges from about 10 kDa to about 5000 kDa. In some embodiments, the molecular weight of a globular protein may range from about 50 kDa to 500 kDa. In some embodiments, the molecular weight of a globular protein may range from about 50 kDa to 200 kDa. In some embodiments, the molecular weight of a globular protein may range from about 50 kDa to 100 kDa.

According to one embodiment, the secondary binding protein or peptide is a monoclonal antibody, or a fragment thereof, capable of binding to at least a section of said at least one cytokine comprised in the immunocytokine, the antibody or fragment comprising
(i) a VH domain comprising a framework and a set of complementarity determining regions HCDR1, HCDR2 and HCDR3, and
(ii) a VL domain comprising a framework and a set of complementarity determining regions LCDR1, LCDR2 and LCDR3

wherein, HCDR3 comprises the amino acid sequence set forth in SEQ ID NO: 6 or SEQ ID No 15, with optionally three or fewer amino acid substitutions.

SEQ ID NO 6 is the HCDR3 of EKH3 as discussed herein, while SEQ ID NO 15 is the HCDR3 of PLG5 as discussed herein.

According to one embodiment thereof, LCDR3 comprises the amino acid sequence set forth in SEQ ID NO: 9 or 18, with optionally three or fewer amino acid substitutions, SEQ ID NO 9 is the LCDR3 of EKH3 as discussed herein, while SEQ ID NO 18 is the LCDR3 of PLG5 as discussed herein.

According to one embodiment thereof,
a) HCDR1 comprises the amino acid sequence set forth in SEQ ID NO: 4 or 13; with optionally three or fewer amino acid substitutions,
b) HCDR2 comprises the amino acid sequence set forth in SEQ ID NO: 5 or 14; with optionally three or fewer amino acid substitutions,
c) LCDR1 comprises the amino acid sequence set forth in SEQ ID NO: 7 or 16; with optionally three or fewer amino acid substitutions, and/or
d) LCDR2 comprises the amino acid sequence set forth in SEQ ID NO: 8 or 17; with optionally three or fewer amino acid substitutions.

SEQ ID NO 4 is the HCDR1 of EKH3, SEQ ID NO 5 is the HCDR2 of EKH3, SEQ ID NO 7 is the LCDR1 of EKH3, SEQ ID NO 8 is the LCDR2 of EKH3, SEQ ID NO 13 is the HCDR1 of PLG5, SEQ ID NO 14 is the HCDR2 of PLG5, SEQ ID NO 16 is the LCDR1 of PLG5, and SEQ ID NO 17 is the LCDR2 of PLG5.

It is applicant's understanding that the definition of the antibody according to the invention by
a) its HCDR3,
b) a combination of LCDR3 and HCDR3, or,
c) a combination of all 6 CDRs

meets all requirements with regard to enablement or written description, mainly due to the functional limitation that the antibody or fragment must bind to at least a section of said at least one cytokine comprised in the immunocytokine

According to still another embodiment, the antibody or fragment thereof comprises
a heavy chain variable domain VH having the sequence set forth in SEQ ID NO: 1 or 10, and
a light chain variable domain VL having the sequence set forth in SEQ ID NO: 2 or 11.

SEQ ID NO 1 is the VH of EKH3, SEQ ID NO 2 is the VL of EKH3; SEQ ID NO 10 is the VH of PLG5, SEQ ID NO 11 is the VL PLG5.

According to still another embodiment the antibody or fragment thereof adopts a format selected from the group consisting of:
IgG
a Fab fragment, a F(ab′)2 fragment, and/or
a Fv (variant fragment), a scFv (single-chain variant fragment) or a scFv-Fc

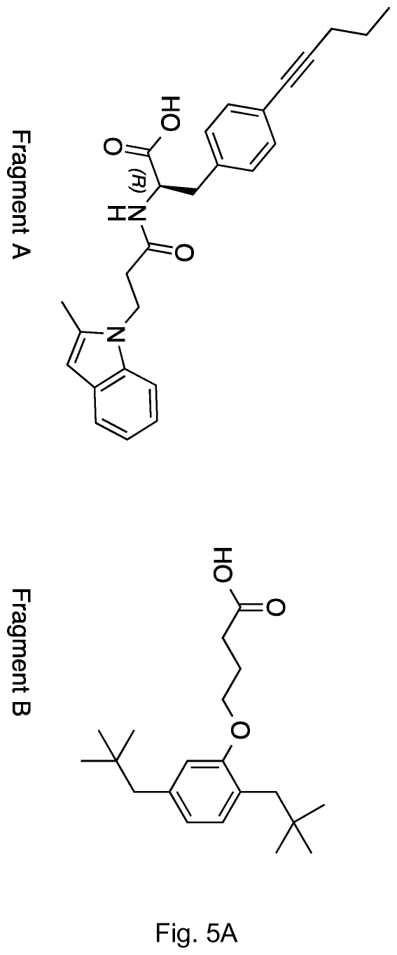

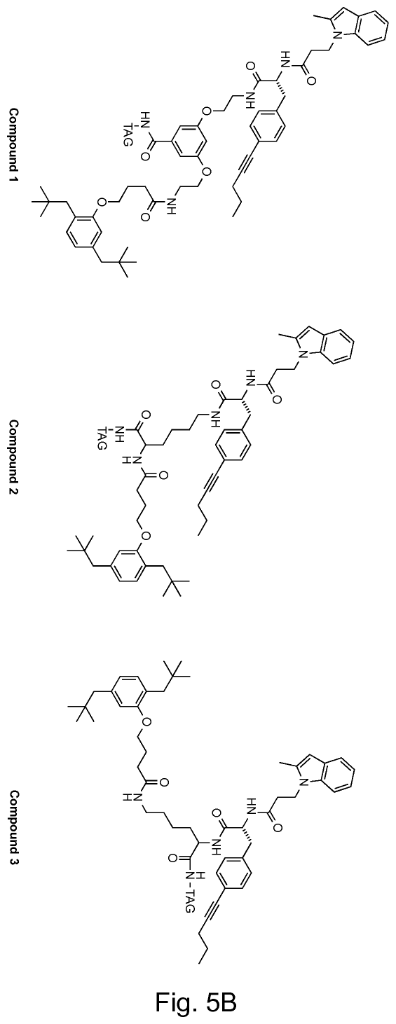

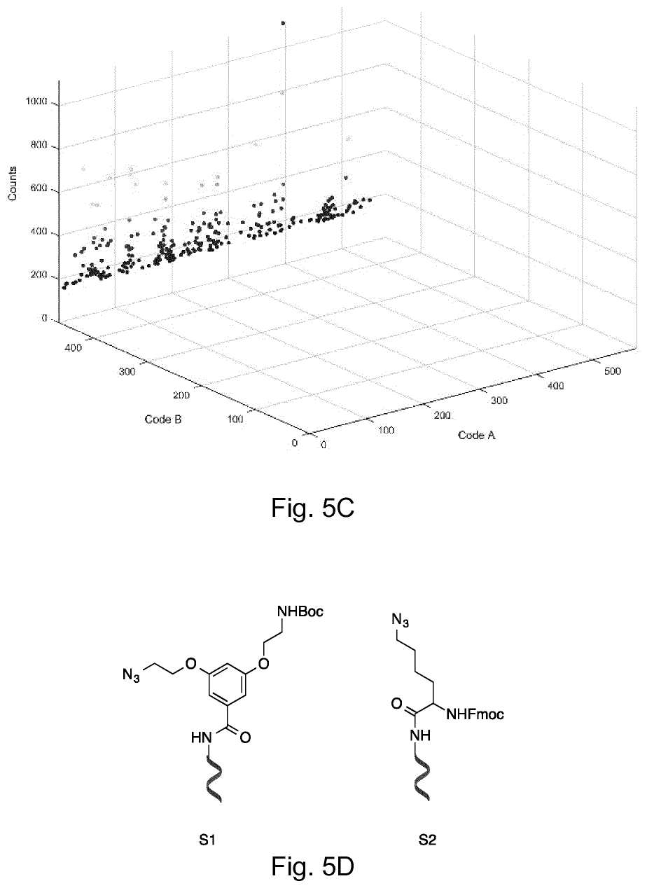

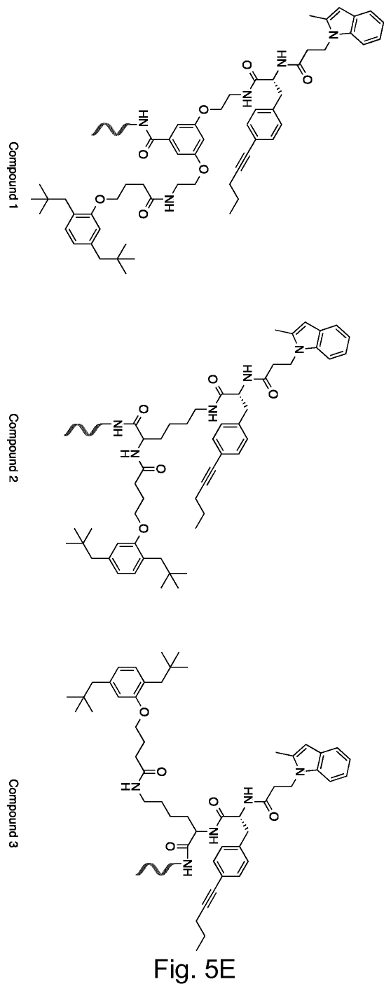

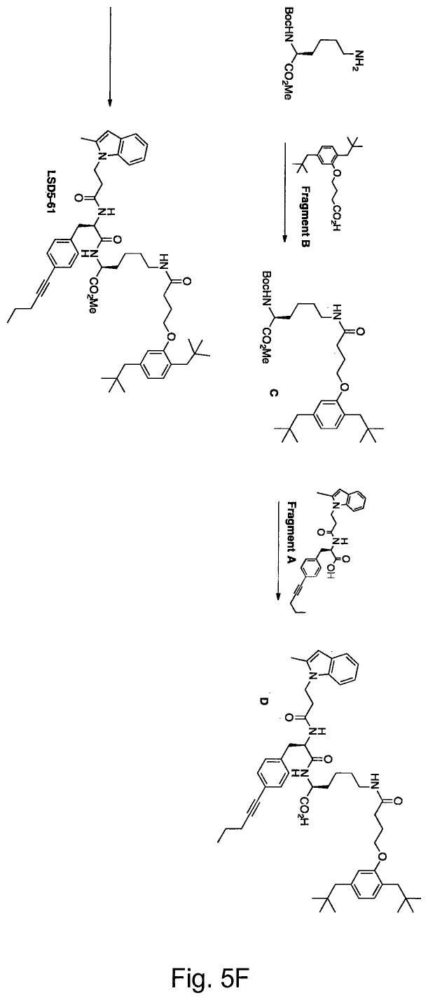

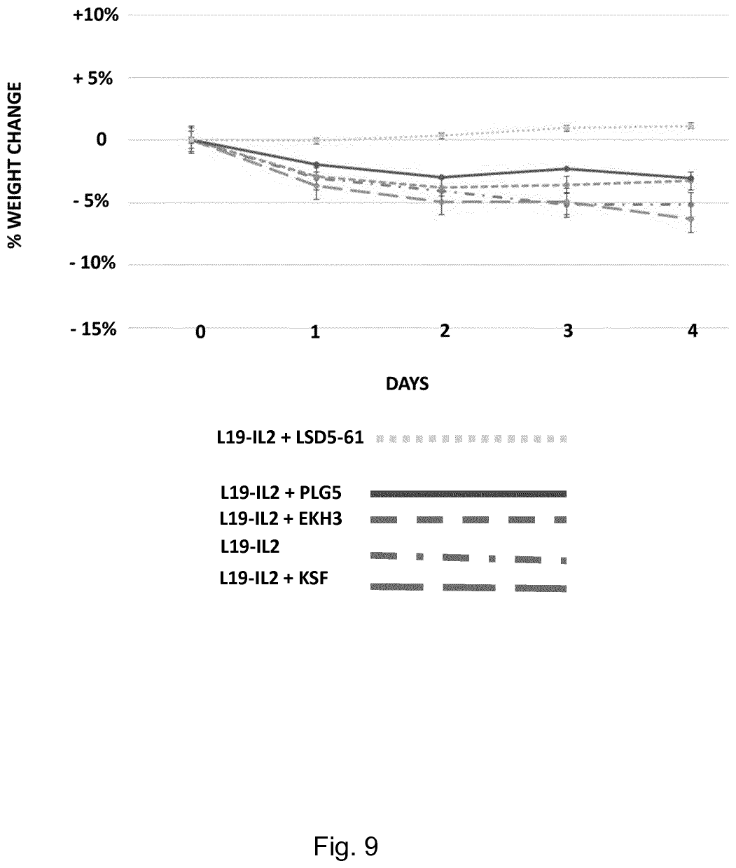

According to another embodiment the small molecule is a methylindole derivative. Such derivatives are shown in FIG. 5, and bind, preferably, to conjugates comprising IL-2. According to yet another embodiment, the small molecule is LSD5-61. This molecule is as well shown in FIG. 5, and its performance in masking L19-IL2 is shown in FIG. 9.

In a preferred embodiment the secondary binding molecule, or masking molecule, does not extravasate from the circulation into the tumor, or into the perivascular tumor space. The masking activity does preferably takes place only when the immunocytokine still is outside the tumor or outside its perivascular space.

In order to attain this goal, the secondary binding molecule, or masking molecule, can be suitably conjugated to an entity that reduces cell membrane permeation, or comprise a moiety that binds to an entity that reduces cell membrane permeation.

Such entity can e.g. be a large protein, like albumin. In such case, the secondary binding molecule, or masking molecule, can be equipped with a binding moiety that binds such protein. Preferred albumin binding moieties which can be conjugated to the masking molecule are for example disclosed in WO2008/053360.

Such entity can also be a large organic polymer, like polyethylene glycole or a sugar, to which the secondary binding molecule, or masking molecule, is conjugated.

Another way to attain this goal is to increase the polarity of the secondary binding molecule, or masking molecule, by providing charged functional groups. The different measures discussed above to reduce extravasation can also be combined.

According to one particular embodiment of the invention, it is provided that
a) the primary binding protein or peptide binds a cancer-related target, and/or
b) the cytokine is an inflammatory cytokine.

As used herein, the term “cancer-related target” relates to a cellular or molecular target that is directly or indirectly involved, or implicated, with the formation of a neoplastic disease, or an increased risk thereof. As used herein, the term “inflammatory cytokine” relates to cytokines that are important in cell signaling and promote systemic inflammation. Examples for cancer-related targets and inflammatory cytokines are disclosed elsewhere herein.

According to one particular embodiment of the invention, it is provided that at least one secondary binding molecule is smaller than, or equally sized as, the primary binding protein or peptide. The sizing refers to at least one parameter selected from the group consisting of molecular weight or diameter. This means for example that secondary binding molecules, which have a smaller size than the conventional IgG format. This includes, for example, Fab fragments, F(ab′)2 fragments, domain antibody (dAb) fragments, diabodies, or scFv fragments, as well as peptides, aptamers or small molecules.

The following table gives a rough overview about typical molecular weights of different types of binding molecules:

TABLE 1

| Type | Molecular weight |
|---|---|
| IgG | 150 kDa |
| Fab | 50 kDa |
| F(ab′)2 | 100 kDa |
| dAb | 12-15 kDa |
| scFv | 25 kDa |
| Peptides ≤ 50 AA | ≤5.5 kDa |
| Aptamers (typically 15-50 nucleotides) | 5-15 kDa |
| Small molecules | ≤2.5 kDa |

In the following table, some preferred target/cytokine combinations are shown:

TABLE 2
suitable target-cytokine combinations

| Target- | | | |
|---|---|---|---|
| for 1^{st} binding | Cytokine | | |
| protein or peptide | TNFα | IL2 | IL12 |
| EDB | X | X | X |
| EDA | X | X | X |
| IIICS | X | X | X |
| TnC-A1 | X | X | X |
| TnC-C | X | X | X |
| TnC-D | X | X | X |

As regards the targets shown in table 2, the applicant has already available suitable antibodies shown in the following table:

TABLE 3
suitable antibodies that may form part of the
immunocytokines of the invention

| Target | Antibody | Disclosed in: |
|---|---|---|
| EDB | L19 | WO2003/076469 |
| EDA | F8 | WO2008/120101 |
| IIICS | SW01 | WO2016/016265 |
| TnC-A1 | F16 | WO2006/050834 |
| TnC-C | G11 | Silacci et al. (2006) PEDS, 19, 471-8 |
| TnC-D | CPR01.1 | WO2017/097990 |

These antibodies have been coexpressed, as a fusion protein, together with suitable cytokines. The general method to make such immunocytokines, and examples of suitable antibody—cytokine combinations have been disclosed, inter alia, in Pasche and Neri, 2013, Clin Pharmacol; 5 (Suppl 1): 29-45.

As regards the cytokines shown in table 2, it is to be understood that these cytokines can be mono- or multimeric. Second binding proteins or peptides binding to these cytokines do preferably bind the epitopes defined in table 4.

TABLE 4
Preferred binding epitopes of some cytokines
that can be used in the present invention

| cytokine | structure | potential epitope to be masked |
|---|---|---|
| IL2 | monomer | Epitope bound by IL2R subunit CD25 |
| IL12 | Heterodimer comprising | IL12 p35, as e.g., bound by IL12-R |
| | p35 and p40 | subunit β2 |
| TNFα | monomeric | same epitopes as bound by anti-TNFα |
| | | biologics, like adalimumab, infliximab |
| | | etanercept, certolizumab or golimumab |

According to one other embodiment of the invention, at least one secondary binding molecule has an affinity towards the cytokine which is essentially equal as, or similar to, the affinity the respective receptor has to the cytokine.

The term “affinity”, as used herein, refers to the binding strength a binder has to its target. Usually, such affinity is expressed by means of the dissociation constant K_{D} [M], which is an equilibrium constant for the dissociation of an antibody-target complex into its components. It is calculated as the ratio k_{off}/kₒₙ. K_{D} and affinity are inversely related, meaning that a low K_{D} indicates a high affinity, while a high K_{D} indicates a low affinity. The following table 5 gives an overview of typical affinities or antibodies for clinical or diagnostic use:

TABLE 5
Affinity ranges of antibodies

| K_{D} value | alias | Relative affinity |
|---|---|---|
| 10⁻⁴ to 10⁻⁶M | Micromolar (μM) | small |
| 10⁻⁷ to 10⁻⁹M | Nanomolar (nM) | medium |
| 10⁻¹⁰ to 10⁻¹²M | Picomolar (pM) | high |
| 10⁻¹³ to 10⁻¹⁵M | Femtomolar (fM) | very high |

Once the immunocytokine has been administered, the secondary binding molecule will gradually dissociate from the immunocytokine, and partially be excreted. This means that gradually, more immunocytokine will become visible for its receptor, hence contributing to an increased pharmacological effect at the site of disease.

In case the affinity of the secondary binding molecule to the cytokine would be substantially higher than the affinity of the receptor thereto, this would mean that the immunocytokine would only slowly develop its pharmacological effect, if at all. In case the affinity of the secondary binding molecule to the cytokine would be substantially lower than the affinity of the receptor thereto, this would mean that the secondary binding molecule would dissociate immediately after administration, hence reducing the protective effect and developing systemic effect, which again would cause side effects.

As used herein, an affinity which is “essentially equal” as, or “similar to”, another affinity means that the two KD may differ from one another not more than 1 order of magnitude. Preferably, they differ from one another not more than 200%, more preferably not more than 100%, more preferably not more than 50% and even more preferably not more than 25% In the case of L19-IL2, the dissociation constant (K_{D}) between IL2 and its receptor, CD25, is about 10⁻⁸ M. Therefore, a secondary binding molecule is preferred which has a dissociation constant (K_{D}) which is in the same range.

The following table shows some typical values for affinities which are “essentially equal” as, or “similar to”, another affinity, as set forth above.

TABLE 6

| | 1 order of | | | | | | | | | |
|---|---|---|---|---|---|---|---|---|---|---|
| | magnitude | | 200% | 200% | 100% | | 50% | | 25% | |
| | affinity | affinity | affinity | affinity | affinity | affinity | affinity | affinity | affinity | affinity |
| _{D} value | lower | higher | lower | higher | lower | higher | lower | higher | lower | higher |
| 10⁻⁰⁴ | 10⁻⁰³ | 10⁻⁰⁵ | 3 × 10⁻⁰⁴ | 3.33 × 10⁻⁰⁵ | 1.50 × 10⁻⁰⁴ | 5 × 10⁻⁰⁵ | 2 × 10⁻⁰⁴ | 6.67 × 10⁻⁰⁵ | 1.33 × 10⁻⁰⁴ | 8 × 10⁻⁰⁵ |
| 10⁻⁰⁵ | 10⁻⁰⁴ | 10⁻⁰⁶ | 3 × 10⁻⁰⁵ | 3.33 × 10⁻⁰⁶ | 1.50 × 10⁻⁰⁵ | 5 × 10⁻⁰⁶ | 2 × 10⁻⁰⁵ | 6.67 × 10⁻⁰⁶ | 1.33 × 10⁻⁰⁵ | 8 × 10⁻⁰⁶ |
| 10⁻⁰⁶ | 10⁻⁰⁵ | 10⁻⁰⁷ | 3 × 10⁻⁰⁶ | 3.33 × 10⁻⁰⁷ | 1.50 × 10⁻⁰⁶ | 5 × 10⁻⁰⁷ | 2 × 10⁻⁰⁶ | 6.67 × 10⁻⁰⁷ | 1.33 × 10⁻⁰⁶ | 8 × 10⁻⁰⁷ |
| 10⁻⁰⁷ | 10⁻⁰⁶ | 10⁻⁰⁸ | 3 × 10⁻⁰⁷ | 3.33 × 10⁻⁰⁸ | 1.50 × 10⁻⁰⁷ | 5 × 10⁻⁰⁸ | 2 × 10⁻⁰⁷ | 6.67 × 10⁻⁰⁸ | 1.33 × 10⁻⁰⁷ | 8 × 10⁻⁰⁸ |
| 10⁻⁰⁸ | 10⁻⁰⁷ | 10⁻⁰⁹ | 3 × 10⁻⁰⁸ | 3.33 × 10⁻⁰⁹ | 1.50 × 10⁻⁰⁸ | 5 × 10⁻⁰⁹ | 2 × 10⁻⁰⁸ | 6.67 × 10⁻⁰⁹ | 1.33 × 10⁻⁰⁸ | 8 × 10⁻⁰⁹ |
| 10⁻⁰⁹ | 10⁻⁰⁸ | 10⁻¹⁰ | 3 × 10⁻⁰⁹ | 3.33 × 10⁻¹⁰ | 1.50 × 10⁻⁰⁹ | 5 × 10⁻¹⁰ | 2 × 10⁻⁰⁹ | 6.67 × 10⁻¹⁰ | 1.33 × 10⁻⁰⁹ | 8 × 10⁻¹⁰ |
| 10⁻¹⁰ | 10⁻⁰⁹ | 10⁻¹¹ | 3 × 10⁻¹⁰ | 3.33 × 10⁻¹¹ | 1.50 × 10⁻¹⁰ | 5 × 10⁻¹¹ | 2 × 10⁻¹⁰ | 6.67 × 10⁻¹¹ | 1.33 × 10⁻¹⁰ | 8 × 10⁻¹¹ |
| 10⁻¹¹ | 10⁻¹⁰ | 10⁻¹² | 3 × 10⁻¹¹ | 3.33 × 10⁻¹² | 1.50 × 10⁻¹¹ | 5 × 10⁻¹² | 2 × 10⁻¹¹ | 6.67 × 10⁻¹² | 1.33 × 10⁻¹¹ | 8 × 10⁻¹² |
| 10⁻¹² | 10⁻¹¹ | 10⁻¹³ | 3 × 10⁻¹² | 3.33 × 10⁻¹³ | 1.50 × 10⁻¹² | 5 × 10⁻¹³ | 2 × 10⁻¹² | 6.67 × 10⁻¹³ | 1.33 × 10⁻¹² | 8 × 10⁻¹³ |
| 10⁻¹³ | 10⁻¹² | 10⁻¹⁴ | 3 × 10⁻¹³ | 3.33 × 10⁻¹⁴ | 1.50 × 10⁻¹³ | 5 × 10⁻¹⁴ | 2 × 10⁻¹³ | 6.67 × 10⁻¹⁴ | 1.33 × 10⁻¹³ | 8 × 10⁻¹⁴ |
| 10⁻¹⁴ | 10⁻¹³ | 10⁻¹⁵ | 3 × 10⁻¹⁴ | 3.33 × 10⁻¹⁵ | 1.50 × 10⁻¹⁴ | 5 × 10⁻¹⁵ | 2 × 10⁻¹⁴ | 6.67 × 10⁻¹⁵ | 1.33 × 10⁻¹⁴ | 8 × 10⁻¹⁵ |
| 10⁻¹⁵ | 10⁻¹⁴ | 10⁻¹⁶ | 3 × 10⁻¹⁵ | 3.33 × 10⁻¹⁶ | 1.50 × 10⁻¹⁵ | 5 × 10⁻¹⁶ | 2 × 10⁻¹⁵ | 6.67 × 10⁻¹⁶ | 1.33 × 10⁻¹⁵ | 8 × 10⁻¹⁶ |

According to one other embodiment of the invention, at least one secondary binding molecule has an affinity towards the cytokine which is smaller than, or equal as, the affinity the primary binding protein or peptide has to its target.

In an embodiment wherein the secondary binding molecule has an affinity towards the cytokine which is smaller than, or equal as, the affinity the primary binding protein or peptide has to its target, the secondary binding molecule will gradually dissociate from the cytokine part of the immunocytokine in vivo, but faster than the primary binding protein or peptide will dissociate from its target. Hence, after the complex comprising the immunocytokine and the secondary binding molecule has reached its in vivo target, the product will progressively gain therapeutic activity due to gradual dissociation of the secondary binding molecule.

In the case of L19-IL2, for example, the dissociation constant (K_{D}) of the binding between L19 and EDB is in the far nanomolar range (10⁻⁸ M). Hence, a secondary binding molecule is preferred which has a dissociation constant (K_{D}) in the near nanomolar range (10⁻⁸-10⁻⁷ M) or in the micromolar range when binding to IL2.

In order to achieve a transient inhibition of cytokine activity, the kinetic dissociation constant of monomeric ligands is particularly important, in order to predict duration of effect. The kinetic dissociation constant k_{off} of the masking molecule is related to the half-life of the complex T_{1/2}^{off} by the relation:

T_{1/2}^{off}=ln 2/k_{off}

Thus, a masking molecule with k_{off}=10⁻³ s⁻¹ will correspond to a half-life of the complex (in conditions of irreversible dissociation) of approx. 11 min., while a k_{off}=10⁻⁴ s⁻¹ will correspond to a half-life of the complex of approx. 2 hours.

While with routine methods, secondary binding molecules can be selected which have a preferred affinity to a target cytokine, such specific selection is particularly simple when using a binding protein or peptide, in particular an antibody, modified antibody format, antibody derivative or fragment, or antibody-based binding protein. The target affinity of these molecules can be readily modulated, or adjusted, with methods known in the art (Mazor Y et al., PLoS One. 2016; 11(6); Schildbach J F et al. Protein Sci. 1993 February; 2(2):206⁻¹⁴; Yu Y et al. Science Translational Medicine 25 May 2011, Vol. 3, Issue 84, pp. 84; Colby D W et al. Methods Enzymol. 2004; 388: 348-58).

According to another aspect of the invention, a complex comprising the combination according to the above description is provided, in which complex at least one secondary binding molecule is bound to at least one cytokine comprised in the immunocytokine.

According to yet another aspect of the invention, a pharmaceutical composition comprising a complex or combination according to the above description is provided, which composition further has at least one further pharmaceutically acceptable ingredient.

According to yet another aspect of the invention, method of preparing a complex or combination according to the above description is provided, said method comprising the steps of:
a) providing the immunocytokine
b) providing the secondary binding molecule, and
c) mixing the two.

Typically, said mixing step will either be carried out during product manufacturing or shortly before administration of the combination or complex to a patient.

According to one embodiment of the invention, the combination, complex or pharmaceutical composition according to the above description is provided for use in the treatment of a human or animal subject that is
suffering from,
at risk of developing, and/or
being diagnosed for

a disease that is indicated for treatment with an immunocytokine, or for the prevention of such condition.

Said combination, complex or pharmaceutical composition is administered to the human or animal subject in an amount or dosage that efficiently treats the disease.

Alternatively, a corresponding method of treatment is provided.

Preferably, in said combination, use or method the pathologic condition is a neoplastic disease.

Preferred Antigens for the Targeting Antibodies

Splice Isoforms of Fibronectin

Fibronectin (FN) is a multimodular glycoprotein found abundantly in the extracellular matrix (ECM) of various connective tissues. FN regulates a wide spectrum of cellular and developmental functions, including cell adhesion, migration, growth, proliferation and wound healing.

FN is secreted from cells as a dimer consisting of two ˜250 kDa subunits covalently linked by a pair of disulfide bonds near their C-termini. Each monomer of FN consists of three types of homologous repeat subunits termed FNI, FNII and FNIII domains, with binding affinity for various ECM proteins.

FN contains 12 FNI, 2 FNII and 15-17 FNIII domains. Based on solubility and tissue distribution, FN occurs in two principal forms, the soluble plasma FN (pFN) circulating in the blood, and the cellular FN (cFN), which polymerizes into insoluble fibers in the ECM of connective tissues.

In the plasma, the pFN dimer does not polymerize and adopts a compacted conformation. cFN on the other hand is synthesized by various cell types including fibroblasts, smooth muscle cells and endothelial cells.

Even though coded by a single gene, FN exists in multiple isoforms as a result of alternative splicing of the precursor mRNA. Splicing occurs at three sites, including the complete 90 amino acid domain EDA or located between 11FNIII and 12FNIII, the complete 91 amino acid EDB domain located between the 7FNIII and 8FNIII domain, and various portions of the 120 amino acid V (variable) or IIICS (connecting segment) domain present between domains 14FNIII and 15FNIII.

The structural diversity created by the alternative splicing of EDA, EDB and IIICS of the primary FN transcript generates at least 20 different isoforms, some of which are differentially expressed in tumour and normal tissue.

The presence of FN isoforms containing the EDA, EDB and IIICS domains in adulthood is very restricted in normal tissue, but prominent in highly remodeling ECM for example during wound healing, atherosclerosis, liver and pulmonary fibrosis, and in vascular tissue and stroma of many cancer types, making this isoforms ideal target for pharmacodelivery.

EDA Domain of Fibronectin

Expression of the EDA of fibronectin has been reported in tumour cells and in solid tumours at the mRNA level in breast cancer and at the level of isolated protein in fibrosarcoma, rhabdomyosarcoma and melanoma. At the immunohistochemical level, the presence of EDA has been detected in the extracellular matrix (ECM) of odontogenic tumours and hepatocellular carcinoma. In contrast, EDA has been detected in the stroma of malignant breast neoplasms, and in the blood vessels and basement membranes of well-differentiated renal cell carcinoma. However, in less-differentiated renal cell carcinoma and papillary carcinoma of the thyroid EDA has been detected in the blood vessels, basement membranes and tumour stroma. The presence of EDA in the vasculature of gliomas has also been reported. The current applicants have also reported EDA expression in tumor metastases (WO2008/120101) as well as in most types of lung cancers and lymphomas (WO2009/013619).

EDB Domain of Fibronectin

The EDB of fibronectin is one of the best-characterized markers of angiogenesis described so far. EDB is synthesized, secreted, and deposited to the extracellular matrix structures by numerous cell types such as endothelial cells of newly formed blood vessels, myofibroblasts, and, most notably, tumor cells. It can be detected at the abluminal site of endothelial linings of newly formed blood vessels and between stromal structures. Using polyclonal, monoclonal, and recombinant antibodies for antigen detection by immunohistochemistry, EDB expression can be demonstrated in a variety of human tissues. EDB expression is tightly controlled and appears to be restricted to embryonic tissues, a few normal adult organs, and wound healing. In vitro studies have yielded contradictory results, and studies with in vivo models on single deletion of EIIIA or EIIIB have failed to provide significant insight into the possible functions of these splice variants. In fact, EIIIA-null or EIIIB-null mice are viable, are fertile, and maintain normal physiological angiogenesis after birth. Nevertheless, EIIIA/EIIIB double-null embryos display multiple cardiovascular defects, thus indicating a crucial involvement in angiogenesis and in cardiovascular development of the EIIIA- and EIIIB-containing splice variants of FN.

Although physiologic expression of EDB is rare in healthy adults, it can occur in chronic pathological conditions associated with new blood vessel formation such as ocular-retinal diseases, severe atherosclerosis, and inflammatory rheumatoid disease. EDB is abundant in tissues of almost all human solid cancers, irrespective of histotype. EDB expression was also found in the majority of lymphoma-infiltrated tissue samples from various Non-Hodgkin lymphoma patients.

IIICS Domain of Fibronectin

Much of the information relating to the expression of the IIICS isoform of fibronectin in healthy and diseased tissues derives either from mRNA studies or from studies with monoclonal antibodies (antibodies FDC-6 and X18A4). These antibodies were generated by hybridoma technology following immunization with fibronectin and immunosuppression with cyclophosphamide. Antibody FDC-6 binds to a specific O-linked N-acetygalactosaminylated hexapeptide epitope within the fibronectin type III connecting segment (IIICS).

However, since the antibody requires both the peptide backbone and the carbohydrate moiety to recognize the epitope, it is not suitable for targeting application especially when cross-reactivity between species is needed. Antibody X18A4 recognizes a different IIICS region than FDC-6, but the binding epitope has never been fully characterized: the main application for antibody X18A4 is related to the detection of oncofetal fibronectin in the cervix of pregnant women to predict preterm labour. There is evidence that IIICS expression is modulated in rheumatoid arthritis and osteoarthritis: in particular, it seems that the isoform 89V (CS1) is up-regulated in inflammation.

Splice Isoforms of Tenascin

Tenascin-C (TNC) is a complex multifunctional protein, which has been shown to promote cell migration, inhibit focal contact formation, promote angiogenesis and, in some systems, act as a cell survival. Each TNC subunit consists of an N-terminal tenascin assembly region, 14.5 epidermal growth factor-like (EGF) repeat domains, a variable number of fibronectin type III-like repeats (FN III) and a C-terminal fibrinogen-like domain. Multiple isoforms of TNC can be generated through alternative splicing of nine FN III repeats between conserved repeats 5 and 6 (exons 9 and 17) at the pre-mRNA level and these may have differing effects. For example, in the developing mouse central nervous system, up to 27 distinct splice variants have been identified and are expressed in a strict temporal-spatial manner supporting a role for these variants in specific neurone-glia interactions. A number of studies have shown that specific functions are mediated by distinct domains of TNC and there is growing evidence to indicate that the biological function of TNC is dependent on the splicing pattern. The changes in the pattern of TNC isoform expression have been described in a number of malignancies, the nature of which appears to be tumour-type specific.

Traditionally the so called “large” isoform of tenascin-C putatively comprises all alternatively spliced domains A1, A2, A3, A4, B, AD, C, D, while in the “small” isoform of tenascin-C these domains were absent.

A1 Domain of Tenascin-C

The alternatively spliced A1 domain of Tenascin-C is virtually undetectable in most of normal organs (except for the placenta and the endometrium in the proliferative phase), whereas it strongly reacts with neovascular and stromal components of many human cancers. Strong expression of domain A1 is typically observed around the vascular structures, as well as in the invasion fronts, of breast cancer, colorectal cancer, gliomas, renal cell carcinoma, melanoma, head & neck cancer etc. . . . . Interestingly, the expression of Tenascin-C in the stroma of tumors is associated with a poor prognosis.

C Domain of Tenascin-C

The alternatively spliced C domain of Tenascin-C was first described by the current applicants in WO2000/063699. The immunohistochemical analysis conducted using an antibodies specific for domain C of TN-C, confirmed that domain C cannot be found in normal adult tissues.

Conversely, high levels of domain C are found in glioblastomas. In this type of tumor, the presence of this TN-C isoform is mainly identified in proximity of vascular structures in areas with high cellular proliferation activity, in the stroma of tumour cell nests, and in proliferating cells. This isoform however can rarely be found in other tumours of the brain. A large presence of domain C is also found in pulmonary neoplasm sections, especially in proximity of vascular structures. Therefore, it can be concluded the C domain is produced by tumour cells, although not by all tumour cells.

D Domain of Tenascin-C

Similar to domain A1 and domain C, the D domain of Tenascin-C is strongly associated to a variety of tumors. Its pattern of expression resembles the one recorded by domain A1 with whom it shares a certain degree of homology.

Preferred Targeting Antibodies

“F8” antibody is the preferred antibody for EDA

This high affinity human antibody was first described by the current applicant in WO2008/120101. It has been shown to target efficiently a variety of tumors.

“L19” antibody is the preferred antibody for EDB

This high affinity human antibody was first described in WO1999/058570. It was further described in “SIP” format in WO2003/076469 and in the radiolabelled version of the “SIP” format in WO2005/023318 by the current applicant. The antibody has been shown to efficiently targeting a variety of tumors.

“SW01” antibody is the preferred antibody for IIICS

This high affinity human antibody was first described in WO2016/016265 by the current applicant. It has been shown to stain efficiently both human and murine tumors sections.

“F16” antibody is the preferred antibody for domain A1 of Tenascin-C

This high affinity human antibody was first described in WO2006/050834 by the current applicant. F16 has been shown to efficiently target in vivo a variety of tumors.

“G11” antibody is the preferred antibody for domain C of Tenascin-C

The capability of the G11 antibody to target tumors efficiently has been described by Silacci et al., Protein Eng Des Sel. (2006) 19, 471-8.

“CPR01.1” antibody is the preferred antibody for domain D domain of Tenascin-C

The ability of this antibody to stain tumor sections has been described by the current applicant in WO2017/097990.

Preferred Cytokines for Conjugation to Targeting Antibodies

Interleukin-2 (IL2)

IL2 is a 15.5-kDa cytokine secreted predominately by Ag-simulated CD4⁺ T cells, but it can also be produced by CD8⁺ cells, NK cells, and activated dendritic cells. IL2 can stimulate cells that express either a trimeric high-affinity IL2 receptor containing the α-, β-, and γ-chains or a low-affinity dimeric receptor consisting of only the β- and γ-chains. In CD8 cells, IL2 can simulate cell growth, as well as differentiation into memory and more terminally differentiated lymphocytes. IL2 is the predominant factor responsible for the maintenance of CD4⁺ regulatory T cells and plays a role in the differentiation of CD4 T cells into a variety of subsets with different T cell functions. Translation of information derived from in vitro and murine tumor models led to the administration of this nonspecific T cell growth factor to patients with cancer and ultimately to the growth and adoptive cell transfer (ACT) of natural or genetically modified autologous human antitumor T cells expanded in vitro in IL2 to treat a variety of cancer types. These findings have had a profound impact on the ability to manipulate the cellular immune system to successfully treat patients with cancer, and they represented the first reproducible demonstrations that manipulations of the immune system could mediate the regression of large human cancers. Unconjugated IL2 is used today to treat patients with kidney cancer and metastatic melanoma, however the high toxicity mainly associated with capillary leak, limits its use in the medical practice.

Interleukin-12 (IL12)

IL12 is a type 1 cytokine that is produced by antigen presenting cells, such as macrophages and CD1c+Dendritic Cells, and acts upon Natural Killer (NK) cells, CD8⁺Cytotoxic T cells, and CD4⁺ T helper cells. Originally called Natural Killer cell stimulating factor, IL12 promotes the cytotoxic activity of NK cells and CD8⁺ T cells and promotes polarization of CD4⁺ T cells towards a type 1 phenotype. Interestingly, human CD4⁺ and CD8⁺ T cells introduced into the xenogeneic environment of humanized mice without IL12 preferentially differentiate into type 2 (IL4+ GATA3+) or mixed type 1 and 2 (IFNG+ TBET+IL4+ GATA3+) subsets. Injection of recombinant human IL12 in mice was able restore differentiation towards a type 1 to improve cytotoxic immunity to a viral challenge. In humans, genetic mutations in IL12p40 and one component of the IL12 receptor, IL12RB1, have been observed in patients with recurrent mycobacterial disease, suggestive of insufficient type 1 cell-mediated immunity. In mice, genetic deletion of other component of the IL12 receptor, IL12RB2, increases susceptibility to spontaneous autoimmunity, B-cell malignancies, and lung carcinomas.

As a single agent, intravenous injection of recombinant IL12 exhibited modest clinical efficacy in a handful of patients with advanced melanoma and renal cell carcinoma. However, one death due to Clostridia perfringens septicemia in the first Phase I study limits interest in the systemic delivery of IL12. As a combination therapy, IL12 has been used as an adjuvant to enhance cytotoxic immunity using a melanoma antigen vaccine or using peptide-pulsed peripheral blood mononuclear cells and to promote NK-cell mediated killing of HER2-positive breast cancer cells in patients treated with trastuzumab.

Tumor Necrosis Factor Alpha (TNFα)

TNFα is a 17 kDa cytokine consisting of 157 amino acids with a 76 amino acid presequence. It can exist in a soluble form or an unprocessed, membrane-bound form (233 amino acids. 26 kDa). TNF-α exists in the biologically-active, physiological form as a homotrimer with a molecular mass of 52 kDa. The shape of the TNF-α homotrimer has the appearance of a triangular cone or bell in which each of the three subunits has a typical jelly roll-β structure and the three subunits are arranged edge to face. In order to establish the location of receptor binding sites on the TNF molecule, random mutagenesis of the gene was performed and inactive molecules were selected on the basis of their cytotoxic activity against murine L-M cells or murine L929 cells. Independent studies demonstrated that the receptor binding sites of TNF-α were located in the lower half of the triangular pyramid in the groove between two subunits.

The multiple activities of TNF-α are mediated through two distinct, high affinity receptors. TNFR55 is a 55 kDa receptor for TNF-α which is ubiquitous, except erythrocytes and unstimulated T cells, and TNFR75 is a 75 kDa receptor which is often more abundant on cells of haemopoietic lineage and is also expressed on endothelium.

Numerous phase I and II trials have been performed with TNF-α being administered intravenously (bolus or infusion), intramuscularly, subcutaneously or intratumorally. In past clinical trials, TNF-α doses have been limited by major side effects with the maximum tolerated dose in the range 150-300 μg/m²/day. The most frequent dose-limiting side effect is hypotension, but other side effects include fatigue, fever, chills, anorexia, headaches, diarrhoea, nausea, vomiting, myalgias, hepatotoxicity, respiratory insufficiency and thrombocytopenia. These side effects are believed to be mainly due to the proinflammatory effects of TNF-α. However, local administration of TNF-α has been used with success. For example some success has been reported for the treatment of patients with melanoma or sarcoma who received high dose TNF-α in combination with IFN-γ and melphalan by isolated perfusion of the involved limbs.

Preferred Antibody-Cytokine Fusions (Immunocytokines)

Preferred Antibody-IL2 Immunocytokines

L19-IL2

L19-IL2 is composed of the human anti-EDB antibody L19 manufactured in scFv format and fused to human interleukin-2 (IL2), which is a pro-inflammatory cytokine. It was first disclosed by the current applicant in WO2001/062298.

L19-IL2 has been proved to be a potent anti-cancer agent in a number of pre-clinical and clinical studies.

The current applicant has further described L19-IL2 and its uses also in WO2007/115837, WO2009/089858, WO2013/010749 and WO2013/045125.

F16-IL2

F16-IL2 is composed of the human antibody F16 manufactured in scFv format (specific to the domain A1 of tenascin-C) and fused to IL2. It has been first disclosed by the current applicant in WO2006/050834. Similarly to L19-IL2, there are many manuscripts reporting the efficacy of F16-IL2 in the treatment of various cancer types.

The current applicant has disclosed F16-IL2 and its uses also in WO2010/078916; WO2011/001276; WO2011/015333;

F8-IL2

F8-IL2 is a product similar to L19-IL2 and to F16-IL2, in which IL2 is conjugated to the F8 antibody. It was first disclosed by the current applicant in WO2010/078945. Although F8-IL2 has never been tested in clinical trials, it has been shown to be an effective immunocytokine in preclinical work.

Preferred Antibody-IL12 Immunocytokines

L19-IL12

L19-IL12 is composed by a first L19 scFv antibody fused to the p35 subunit of IL12 which—in turn—is fused to the p40 subunit of IL12 which—in turn—is fused to a second L19 scFv. It was first disclosed by the current applicant in WO2006/119897.

F8-IL12

F8-IL12 is composed by the p40 subunit of IL12 sequentially fused to the p35 subunit of IL12 which—in turn—are sequentially fused to F8 in diabody configuration. It was first disclosed by the current applicant in WO2013/014149.

Preferred Antibody-TNF Immunocytokines

L19-TNF

L19-TNF is composed by the L19 antibody fused to TNFα which forms a non-covalent homotrimer. It was first disclosed by the current applicant in WO2001/062298. L19-TNF has been studied in different clinical trials.

F8-TNF

Similar to L19-TNF, F8-TNF is an immunocytokine composed by the F8 antibody and TNF. It has been described in Hemmerle et al., Br J Cancer. 2013 Sep. 3; 109(5):1206-13.

Preferred Immunocytokines Featuring Two Distinct Cytokines

Alternatively the immunocytokine may also comprise a targeting antibody fused to two distinct immunocytokines such as IL2-F8-TNF as disclosed in WO2016/180715 or mutants thereof.

Preferred Epitopes of the Cytokines to be Masked

IL2

IL2 mediates its effects by binding to IL2 receptors, which are expressed by lymphocytes. The IL2 receptor has three forms, generated by different combinations of three different proteins, often referred to as “chains”: α (alpha) (also called IL2Rα, CD25), β (beta) (also called IL2Rβ, or CD122), and γ (gamma) (also called IL2Rγ, or CD132); these subunits are also parts of receptors for other cytokines.

By binding to CD25 IL2 exerts a number of unwanted effects including stimulation of immunosuppressive regulatory T cells (Tregs) and contribution to vascular leak syndrome which is the major cause of IL2-associated toxicity in patients.

In a preferred embodiment of this invention the IL2 masking molecule, blocks the epitope of IL2 which binds CD25 therefore inhibiting the binding between the IL2-based immunocytokines and CD25.

IL12

IL12 is a cytokine extensively investigated for its anti-tumor properties. IL12 exerts antitumor activity through IFNγ-dependent and independent mechanisms, which include modulation of the immune system and anti-angiogenesis. Unfortunately, in clinical trials in patients with cancer, systemic i.v. administration of recombinant IL12 not only demonstrated poor efficacy but also caused severe adverse effects. IL12 mediates its biological function by binding to the IL12 receptor which is a heterodimeric receptor formed by the IL12R-β1 and IL12R-β2 that exist primarily on T and NK cells. Naive T cells express IL12Rβ1 but not IL12R-β2, which is critical for the signal transduction downstream of the receptor complex.

In a preferred embodiment of this invention the IL12 masking molecule, blocks the epitope of IL12 which binds IL12R-β2 therefore inhibiting the binding between IL12-based immunocyokines and IL12R-β2.

TNF

The multiple activities of TNF-α are mediated through two distinct, high affinity receptors. TNFR55 is a 55 kDa receptor for TNF-α which is ubiquitous, except erythrocytes and unstimulated T cells, and TNFR75 is a 75 kDa receptor which is often more abundant on cells of haemopoietic lineage and is also expressed on endothelium. TNF-α and its receptors (TNFR55 and TNFR75) are the prototype members of two superfamilies. Twelve receptors have been identified in the TNF receptor superfamily and eight cognate ligands have been discovered thus far in the TNF ligand superfamily.

In a preferred embodiment of this invention, the TNF masking molecule is a TNF blocker approved for human use such as Adalimumab, Infliximab, Certolizumab, Golimumab or Etanercept. In another embodiment of this invention, the TNF masking molecule binds the same epitope recognized by Adalimumab, Infliximab, Certolizumab or Golimumab, but with a lower affinity.

### EXPERIMENTS AND FIGURES

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. Any reference signs should not be construed as limiting the scope.

### EXPERIMENTS

Primary antibodies which are fused to cytokines:

| | Target | Antibody |
|---|---|---|
| 1 | EDB | L19 |
| 2 | EDA | F8 |
| 3 | IIICS | SW01 |
| 4 | TnC-A1 | F16 |
| 5 | TnC-C | G11 |
| 6 | TnC-D | CPR01.1 |

Cytokines to which these antibodies are fused:

IL2
IL12
TNF

Epitopes of cytokines which are masked by suitable antibodies (scFv) or small molecules (methylindole derivative)

| cytokines | Epitope masked in the cytokine |
|---|---|
| IL2 | CD25 |
| IL12 | IL12-R-β2 |
| TNF | Epitope bound by TNF-blockers |

The immunocytokine is produced as a fusion peptide with, e.g., SEQ ID No 19 or 20 (which stand for L19-IL2 and L19-TNF). An expression protocol is for example disclosed in Mårlind et al (2008) (where the expression of F16-IL2 is described, however, this can be transferred to the other immunocytokines referred to herein).

The masking molecule are produced through separate preparations and stored in different vials; for example by phage display of respective scFv antibodies against IL2, IL12 and TNF, or by the method disclosed in Leimbacher et al (2012).

When selecting the masking molecule, care is taken that the K_{D} of the masking molecule (when bound to the cytokine) is equal as, similar to or smaller than, the K_{D} of the cytokine to its receptor.

In the case of L19-IL2, the dissociation constant (K_{D}) of the binding between IL2 and CD25 is in the far micromolar range (10⁻⁸ M). Hence, a masking molecule is preferred which has a dissociation constant in the near micromolar range (10⁻⁸-10⁻⁷ M) or in the micromolar range when binding to IL2.

Shortly (less than 5 minutes) before administration, the immunocytokine and the masking molecule are pre-mixed in a single vial, or directly in the syringe used for administration.

The combination is administered into a xenograft mouse bearing a tumour that is responsive for the respective cytokine tumor bearing animal. See the following table for some examples:

| Immuno- | | |
|---|---|---|
| cytokine | Xenograft model | reference |
| F8-IL2 | WM1552/5 (orthotopic | Moschetta M et al. Cancer Res. |
| | melanoma model) | 2012 Apr. 1; 72(7): 1814-24. |
| | A375M Metastatic | |
| | (melanoma model) | |
| F8-IL2 | Caki-1 (kidney cancer) | Frey K et al, J Urol 2010; 184: |
| | | 2540-8. |
| L19-TNF | WEHI-164 and Sarcoma | Hemmerle T et al. British Journal |
| | 180 (sarcoma) | of Cancer (2013) 109, 1206-1213 |
| L19-IL12 | F9 murine (terato- | Gafner V et al (2006) Int J |
| | carcinoma) | Cancer 119: 2205-22 12 |

Four comparative experiments are being carried out per immunocytokine/masking molecule combination:

(i) immunocytokine alone;

(ii) immunocytokine and the masking molecule having same molarity;

(iii) immunocytokine in combination with a molar excess of the masking molecule;

(iv) masking molecule in combination with a molar excess of the immunocytokine

### EXAMPLES

### Example 1: Preparation of an Anti L19-IL2 Organic Binder

Through dual-pharmacophore DNA-encoded chemical libraries we discovered two fragments that synergize in the binding to IL2 fused to the immunocytokine L19-IL2. The fragments are reported in FIG. 5A.

Fragment A was already reported as weak-micromolar binder and inhibitor of IL2 (Leimbacher et al (2012).

The two fragments were connected using a series of bi-functional scaffolds bearing a proper fluorescent tag for affinity measurements through florescent polarization techniques. The structures of the three compounds tested are reported in FIG. 5B. Affinity constants (K_{D}) for compounds 1-3 measured by florescent polarization are reported in Table 1.

| Compound ID | K_{D} (nM) |
|---|---|
| 1 | 19 ± 7 |
| 2 | 22 ± 8 |
| 3 | 29 ± 12 |

Materials and Methods

General Remarks and Procedures

Synthetic oligonucleotides were purchased from various commercial suppliers and stored as water solutions at −20° C. Chemical compounds were purchased from various commercial suppliers. Enzymes were purchased from various commercial suppliers.

DNA precipitation was performed with the addition of a 5M NaCl and EtOH mixture, −20° C.>4 hours and then centrifugation at 0° C. for 25 minutes at 14000 rpm. The pellet was dried using a SpeedVac and the crude DNA was purified by RP-HPLC on an XTerra® C18 semipreparative using a gradient of eluent A (TEAA 100 mM) and eluent B (TEAA 100 mM in 80% ACN). DNA quantification was determined by measuring the UV absorbance at 260 nM of a water solution on a Thermofisher nanodrop 2000.

The fluorophore used in the binding affinity measurements by fluorescence polarization was BODIPY™ TMR-X NHS Ester (D6117, Thermo Fisher Scientific). The fluorescence anisotropy was measured on a Spectra Max Paradigm multimode plate reader (Molecular Devices) using the Rhodamine-FP filter (Excitation: 535 nm, Emission: 595 nm).

Construction of Encoded Self-Assembling Affinity Maturation Chemical Library

The affinity maturation library consists of two sub-libraries. The 5′ sub-library (sub-library A) carries 550 different compounds at the 5′-end of a single-stranded oligonucleotide containing an individually identifying sequence, specific for each compound. The 3′ sub-library (sub-library B) consists of single compounds coupled to the 3′-end of a complementary single-stranded oligonucleotides and contains a IL2 binding moiety (fragment A, FIG. 5A)

Sub-libraries are mixed in equimolar amounts and hybridized by heating. Klenow fill-in is used to transfer coding information from the 3′-strand to the 5′-strand.

(i) Construction of a Sub-Library of Oligonucleotide-Compound Conjugate Using 5′-Amino-Modified Oligonucleotides (Sub-Library A)

Commercially purchased oligonucleotides carrying a 5′ primary amino group and an individual encoding sequence were coupled to carboxylic acids, acyl chlorides, cyclic anhydride, or isothiocyanates. After coupling, HPLC purification and quality control (LC-ESI-MS), equimolar amounts of encoded compounds were mixed together to generate the desired sub-library A.

(ii) Construction of a Sub-Library of Oligonucleotide-Compound Conjugates Using 3′-Aminomodified, 5′-Phosphorylated Oligonucleotides (Sub-Library B)

Compounds are coupled at 3′-end of a single-stranded oligonucleotide. The 3′-oligonucleotide contains a d-spacer (abasic nucleotide backbone) in order to allow hybridization to sub-library A. The identifying code was added in a subsequential ligation step. The final products were purified and pooled in equimolar amounts in order to yield the final sub-library B.

(iii) Synthesis of Fragment A Oligonucleotide Conjugate

12.5 μL of a 200 mM stock solution of fragment A in DMSO were activated for 30 min at 30° C. with 24 μL 100 mM 1-ethyl-3-(3-dimethylaminopropyl) carbodiimide (EDC) in DMSO and 20 μL 333 mM N-hydroxysulfosuccinimide (S—NHS) in DMSO/H20 2:1, in 200 μL DMSO and subsequently reacted overnight at 37° C. with 5 nmol of amino-modified oligonucleotide dissolved in 50 μL 500 mM trimethylamine/hydrogen chloride (TEA/HCl pH 10). After reaction, the DNA conjugate was precipitated, HPLC purified and analyzed by LC-ESI-MS.

(iv) Encoding by Ligation

100 pmol of compound-oligonucleotide conjugates (example 1.2.1) (20 μL of 5 μM solution), 13 μL of a 10 μM coding oligonucleotide (130 pmol), 7 μL of a 28.7 μM chimeric RNA/DNA adapter oligonucleotide, 10 μL NEB 10× ligase buffer and 50 μL H20 were mixed and heated up to 90° C. for 2 min. Then the mixture was passively cooled down to 22° C. (hybridization). Afterwards, 2 μL NEB ligase was added. Ligation was performed at 16° C. for 14 hours. The ligase was inactivated for 15 min at 70° C.

Chimeric RNA/DNA adaptor was performed by adding to 50 pmol of the ligate compound (50 μL), 5.7 μL of 10× reaction buffer and 1.0 μL of RNase HII. The reaction was carried at 37° C. overnight. The conjugates were then purified by Smartpure Eurogentech purification kit (SK-PCPU-100).

(v) Preparation of the Affinity Maturation DNA-Encoded Library

5 pmol of pooled 3′ compound oligo nucleotide conjugates (Sub-library B, example 1.2), 5 pmol of pooled 5′ compound conjugate (Sub-library A, example 1.1), 5 μL of 10×NEB2 reaction buffer and water for a total volume of 47.5 μL were mixed and heated up to 90° C. for 2 min, then cooled to 22° C. for hybridization. 2 μL 5 mM dNTPs and 0.5 μL NEB Klenow polymerase were added and the sample was incubated at 25° C. for 60 min. After QC (TBE-Urea 15% gel, detection with SybrGreen), the obtained encoded self-assembling chemical library was directly used for target-based selections.

(vi) Affinity Screening of Affinity Maturation Library Against L19-IL2

Affinity selections were performed using a KingFisher magnetic particle processor from Thermo Fisher Scientific.

Magnetic beads streptavidin-coated (10 μL, Dynabeads MyOne Streptavidin C1, Invitrogen) were incubated with biotinylated L19-IL2 (100 μL, 1 μM in PBS) for 30 minutes. 2 rounds of washing with 200 μL PBS with 0.05% Tween-20 and 1 mM Biotin were then performed in order to remove the protein excess. An additional round of washing with 200 μL PBS with 0.05% Tween-20 was performed.

Protein-coated beads were then incubated with ESAC affinity maturation library (Example 1.3) (0.5 pmol in 100 μL PBS with 0.05% Tween-20 and herring Sperm as blocking agent) for 1 h with continuous mixing. Unbounded library members were then removed by washing 5 times with 200 μL PBS 0.05% Tween-20. Beads carrying bond library members were resuspended in elution buffer (100 μL Tris-HCl, pH 8.5) and the binding DNA-compound conjugates released from the beads by heat denaturation of the streptavidin and L19-IL2 (95° C., 10 min). The eluted DNA was amplified by PCR, introducing at the same time additional selection specific primers. The selection result was submitted to Illumina high-throughput DNA sequencing and sequencing output was decoded and visualized by an in-house developed program (FIG. 5C)

(vii) On-DNA Re-Synthesis of the Binding Conjugated for Hit-Validation Purpose

Analysis of high-throughput DNA sequencing data (FIG. 5C) revealed a highly enriched pharmacophore pair Fragment A-Fragment B (FIG. 5A). To confirm these results, Fragment A-Fragment B were connected using a series of bi-functional scaffolds (S1 and S2) bearing a 12-mer DNA tag. (FIG. 5D)

Screening of Compound 1 (FIG. 5B)

(i) Coupling of S1 to the 12-Mer DNA Tag.

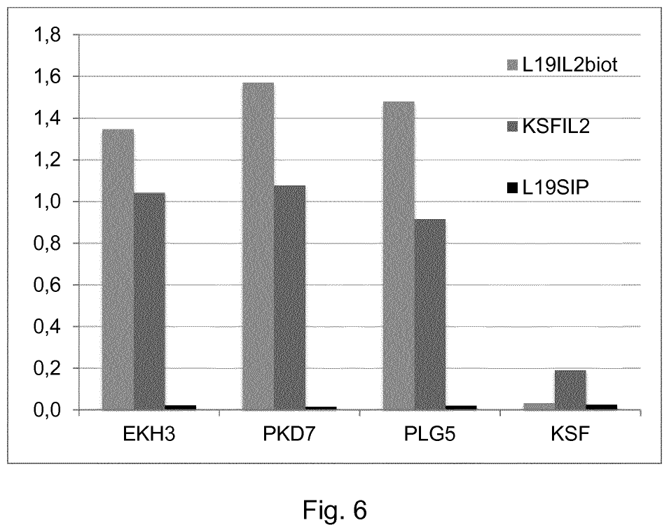

A 200 mM stock solution of S1 (FIG. 6) in DMSO (12.5 μL, 2.50 μmol, 1.24 mg) was diluted with DMSO (225 μL) in a 2 mL Eppendorf tube. A 333 mM stock solution of sulfo-NHS in DMSO-H₂O 2:1 (20 μL, 6.7 μmol, 1.4 mg) was added, followed by a 100 mM DMSO solution of EDC (24 μL, 2.4 μmol, 0.37 mg). The resulting mixture was shaken on a mechanical shaker at 30° C. for 20 minutes. In the meantime, the 12-mer DNA tag (100 nmol) was dissolved in 100 mM MOPS/1M NaCl, pH 8 buffer (50 μL). The DMSO mixture containing the activated S1 was combined with the 12-mer DNA solution and the resulting mixture was shaken at 37° C. for 14 hours. The DNA was isolated by precipitation.

(ii) Boc Deprotection.

The crude DNA pellet from the previous step was dissolved in 500 mM borate buffer at pH 9.4 (100 μL) and 1M MgCl₂ solution (0.3 μmol) was added. The resulting mixture was shaken at 90° C. for 16 hours. The reaction mixture was cooled to room temperature and the DNA was isolated by precipitation and HPLC purification.

(iii) Coupling of Fragment A.

The DNA obtained in the Boc deprotection step was dissolved in 100 mM MOPS/1M NaCl, pH 8 buffer (50 μL). A 200 mM stock solution of fragment A (FIG. 5) in DMSO (12.5 μL, 2.50 μmol, 1.24 mg) was diluted with DMSO (225 μL) in a 2 mL Eppendorf tube. A 333 mM stock solution of sulfo-NHS in DMSO-H₂O 2:1 (20 μL, 6.7 μmol, 1.4 mg) was added, followed by a 100 mM DMSO solution of EDC (24 μL, 2.4 μmol, 0.37 mg). The resulting mixture was shaken on a mechanical shaker at 30° C. for 20 minutes. The DMSO solution containing the activated fragment A was combined with the DNA solution and the resulting mixture was shaken at 37° C. for 14 hours. The DNA was isolated by precipitation.

(iv) Azide Reduction.

Compound S1-fragment A was dissolved in a 30 mM solution of TCEP-HCl prepared using 500 mM TRIS-HCl at pH 7.4 (100 μL). The resulting solution was shaken in a 2 mL Eppendorf tube at 30° C. for 14 hours. The DNA was isolated by precipitation and used directly for the coupling of fragment B.

(v) Coupling of Fragment B.

The crude DNA pellet from the azide reduction step was dissolved in 100 mM MOPS/1M NaCl, pH 8 buffer (50 μL). A 200 mM stock solution of fragment B (FIG. 5A) in DMSO (12.5 μL, 2.50 μmol, 1.24 mg) was diluted with DMSO (225 μL) in a 2 mL Eppendorf tube. A 333 mM stock solution of sulfo-NHS in DMSO-H₂O 2:1 (20 μL, 6.7 μmol, 1.4 mg) was added, followed by a 100 mM DMSO solution of EDC (24 μL, 2.4 μmol, 0.37 mg). The resulting mixture was shaken on a mechanical shaker at 30° C. for 20 minutes. The DMSO solution containing the activated fragment B was combined with the DNA solution and the resulting mixture was shaken at 37° C. for 14 hours. The DNA was isolated by precipitation and HPLC purification. The fractions containing the product were combined and lyophilized to obtain compound 1 (FIG. 5E).

Screening of Compound 2 (FIG. 5B)

(i) Coupling of S2 to the 12-Mer DNA Tag.

A 200 mM stock solution of S2 (FIG. 6) in DMSO (12.5 μL, 2.50 μmol, 1.24 mg) was diluted with DMSO (225 μL) in a 2 mL Eppendorf tube. A 333 mM stock solution of sulfo-NHS in DMSO-H₂O 2:1 (20 μL, 6.7 μmol, 1.4 mg) was added, followed by a 100 mM DMSO solution of EDC (24 μL, 2.4 μmol, 0.37 mg). The resulting mixture was shaken on a mechanical shaker at 30° C. for 20 minutes. In the meantime, the 12-mer DNA tag (100 nmol) was dissolved in 100 mM MOPS/1M NaCl, pH 8 buffer (50 μL). The DMSO mixture containing the activated S2 was combined with the 12-mer DNA solution and the resulting mixture was shaken at 37° C. for 14 hours. The DNA was isolated by precipitation.

(ii) Fmoc Deprotection.

The crude DNA pellet was dissolved in H₂O (100 μL) and triethylamine (7 μL, 0.5 mg, 5 μmol) was added. The resulting mixture was shaken at 37° C. until complete deprotection of the Fmoc group (4 hours) as judged by analysis of the crude reaction mixture by LC-MS. The reaction mixture was cooled to room temperature and the DNA was isolated by precipitation.

(iii) Coupling of Fragment B

The DNA obtained in the fmoc-deprotection step was dissolved in 100 mM MOPS/1M NaCl, pH 8 buffer (50 μL). A 200 mM stock solution of fragment B (FIG. 5A) in DMSO (12.5 μL, 2.50 μmol, 1.24 mg) was diluted with DMSO (225 μL) in a 2 mL Eppendorf tube. A 333 mM stock solution of sulfo-NHS in DMSO-H₂O 2:1 (20 μL, 6.7 μmol, 1.4 mg) was added, followed by a 100 mM DMSO solution of EDC (24 μL, 2.4 μmol, 0.37 mg). The resulting mixture was shaken on a mechanical shaker at 30° C. for 20 minutes. The DMSO solution containing the activated fragment B was combined with the DNA solution and the resulting mixture was shaken at 37° C. for 14 hours. The DNA was isolated by precipitation.

(iv) Azide Reduction

Compound S2-fragment B was dissolved in a 30 mM solution of TCEP-HCl prepared using 500 mM TRIS-HCl at pH 7.4 (100 μL). The resulting solution was shaken in a 2 mL Eppendorf tube at 30° C. for 14 hours. The DNA was isolated by precipitation and used directly for the coupling of fragment A.

(v) Coupling of Fragment A.

The crude DNA pellet from the azide reduction step was dissolved in 100 mM MOPS/1M NaCl, pH 8 buffer (50 μL). A 200 mM stock solution of fragment A (FIG. 5A) in DMSO (12.5 μL, 2.50 μmol, 1.24 mg) was diluted with DMSO (225 μL) in a 2 mL Eppendorf tube. A 333 mM stock solution of sulfo-NHS in DMSO-H₂O 2:1 (20 μL, 6.7 μmol, 1.4 mg) was added, followed by a 100 mM DMSO solution of EDC (24 μL, 2.4 μmol, 0.37 mg). The resulting mixture was shaken on a mechanical shaker at 30° C. for 20 minutes. The DMSO solution containing the activated fragment A was combined with the DNA solution and the resulting mixture was shaken at 37° C. for 14 hours. The DNA was isolated by precipitation and HPLC purification. The fractions containing the product were combined and lyophilized to obtain compound 2. (FIG. 5E)

Screening of Compound 3 (FIG. 5B)

(i) Coupling of S2 to the 12-Mer DNA Tag.

A 200 mM stock solution of S2 (FIG. 6) in DMSO (12.5 μL, 2.50 μmol, 1.24 mg) was diluted with DMSO (225 μL) in a 2 mL Eppendorf tube. A 333 mM stock solution of sulfo-NHS in DMSO-H₂O 2:1 (20 μL, 6.7 μmol, 1.4 mg) was added, followed by a 100 mM DMSO solution of EDC (24 μL, 2.4 μmol, 0.37 mg). The resulting mixture was shaken on a mechanical shaker at 30° C. for 20 minutes. In the meantime, the 12-mer DNA tag (100 nmol) was dissolved in 100 mM MOPS/1M NaCl, pH 8 buffer (50 μL). The DMSO mixture containing the activated S2 was combined with the 12-mer DNA solution and the resulting mixture was shaken at 37° C. for 14 hours. The DNA was isolated by precipitation.

(ii) Fmoc Deprotection

The crude DNA pellet was dissolved in H₂O (100 μL) and triethylamine (7 μL, 0.5 mg, 5 μmol) was added. The resulting mixture was shaken at 37° C. until complete deprotection of the Fmoc group (4 hours) as judged by analysis of the crude reaction mixture by LC-MS. The reaction mixture was cooled to room temperature and the DNA was isolated by precipitation.

(iii) Coupling of Fragment A

The DNA obtained in the Fmoc deprotection step was dissolved in 100 mM MOPS/1M NaCl, pH 8 buffer (50 μL). A 200 mM stock solution of fragment A (FIG. 5A) in DMSO (12.5 μL, 2.50 μmol, 1.24 mg) was diluted with DMSO (225 μL) in a 2 mL Eppendorf tube. A 333 mM stock solution of sulfo-NHS in DMSO-H₂O 2:1 (20 μL, 6.7 μmol, 1.4 mg) was added, followed by a 100 mM DMSO solution of EDC (24 μL, 2.4 μmol, 0.37 mg). The resulting mixture was shaken on a mechanical shaker at 30° C. for 20 minutes. The DMSO solution containing the activated fragment A was combined with the DNA solution and the resulting mixture was shaken at 37° C. for 14 hours. The DNA was isolated by precipitation.

(iv) Azide Reduction

Compound S2-fragment A was dissolved in a 30 mM solution of TCEP-HCl prepared using 500 mM TRIS-HCl at pH 7.4 (100 μL). The resulting solution was shaken in a 2 mL Eppendorf tube at 30° C. for 14 hours. The DNA was isolated by precipitation and used directly for the coupling of fragment B.

(v) Coupling of Fragment B

The crude DNA pellet from the azide reduction step was dissolved in 100 mM MOPS/1M NaCl, pH 8 buffer (50 μL). A 200 mM stock solution of fragment B (FIG. 5A) in DMSO (12.5 μL, 2.50 μmol, 1.24 mg) was diluted with DMSO (225 μL) in a 2 mL Eppendorf tube. A 333 mM stock solution of sulfo-NHS in DMSO-H₂O 2:1 (20 μL, 6.7 μmol, 1.4 mg) was added, followed by a 100 mM DMSO solution of EDC (24 μL, 2.4 μmol, 0.37 mg). The resulting mixture was shaken on a mechanical shaker at 30° C. for 20 minutes. The DMSO solution containing the activated fragment B was combined with the DNA solution and the resulting mixture was shaken at 37° C. for 14 hours. The DNA was isolated by precipitation and HPLC purification. The fractions containing the product were combined and lyophilized to obtain compound 3. (FIG. 5E)

Affinity Determination by Fluorescence Polarization (FP) Measurements

DNA-tagged compounds 1, 2 and 3 (FIG. 5E) were separately incubated and hybridized with an equimolar amount of Fluorescently labelled 8-mer complementary amino-modified locked nucleic acids (LNAs) to form a proper fluorescent structure suitable for fluorescence polarization affinity measurements against L19-IL2 (30 min, 24° C.).

Labelled ligands (25 nM, 25 μL) were incubated at 22° C. for 30 min in a black 384-well plate (Greiner, non-binding) in PBS (pH 7.4) with increasing concentrations of L19-IL2 to a total volume of 75 μl. The fluorescence anisotropy was measured on a Spectra Max Paradigm multimode plate reader (Molecular Devices). Anisotropy values were fitted using KaleidaGraph 4.1.3 (Synergy Software). The results are shown in Table 1.

Synthesis of LSD5-61

After the determination of the Kd by fluorescence polarization, it was decided to synthesize the compound 3 (FIG. 5b) without the DNA tag. The name of Compound 3 without DNA is LSD5-61. The synthesis of LSD5-61 is reported here below and schematically in FIG. 5F.

Synthesis of Compound C:

(S)-Methyl 6-amino-2-((tert-butoxycarbonyl)amino)hexanoate (112 mg, 0.429 mmol, 1.1 eq) was dissolved in 2.5 ml DMF. The acid B (125 mg, 0.390 mmol, producted in house), DIPEA (170 μl, 0.975 mmol, 2.5 eq) and HATU (200 mg, 0.526 mmol, 1.35 eq) were added. The mixture was stirred at rt overnight. The reaction was diluted with H₂O and extracted with Et₂O and then dried with Na₂SO₄. The solvent was removed under reduced pressure the crude residue was purified by flash column chromatography on silica gel (Hexane:EtOAc=3:2) to afford A (161 mg, 73%).

Synthesis of Compound D:

Compound C (161 mg, 0.284 mmol) was dissolved in 1 ml DCM in a 10 ml round bottomed flask. The mixture was cooled at 0° C. in an ice bath and TFA (500 μl, 6.50 mmol, 20.0 eq) was added. Then the solution was stirred at room temperature for 2 h. The solvent was evaporated and then co-evaporated with toluene (×3) to obtain the deprotected product in quantitative yield. The crude material (164 mg, 0.284 mmol) was dissolved in 2.5 ml DMF. The Fragment A (154 mg, 0.369 mmol, 1.3 eq) DIPEA (193 μl, 1.11 mmol, 3.0 eq), HATU (146 mg, 0.383 mmol, 1.4 eq) were added. The mixture was stirred at room temperature overnight. The reaction was diluted with H₂O and extracted with Et₂O and then dried with Na₂SO₄. The solvent was removed under reduced pressure the crude residue was purified by flash column chromatography on silica gel (Hexane:EtOAc=1:3) to afford E (170 mg, 69%).

Synthesis of LSD5-61

Compound D (100 mg, 0.115 mmol) was dissolved in 800 μl THF, 400 μl MeOH and 800 μl 1 M KOH solution was added. The reaction mixture was stirred at room temperature for 4 h. 1 M HCl was added to reach pH 2, then the mixture was extracted with DCM (twice) and dried with Na₂SO₄. The solvent was removed under reduced pressure to obtain 100 mg of LSD5-61 (pure by LC-MS).

### Example 2: Preparation of Anti-IL2 scFv Antibodies

Material & Methods

Antigen Preparation for Antibody Phage Display Selections

The fusion protein antibody (L19)-interleukin 2 (L19-IL2) was used as antigen to isolate human antibody fragments specific to IL2. L19-IL2 was biotinylated using EZ-Link® NHS-Biotin Reagent (Thermo Scientific) in order to attach 2 biotin moieties per L19-IL2 molecule.

Antibody Selection Protocol

Antibody selection was performed essentially as described in Silacci et al (2005). 60 ul of magnetic dynabeads (Invitrogen) were coated with 120 pmol of biotinylated antigen. PHILO1 and PHILO2 libraries were used for antibody selection (WO2010/028791). In order to prevent the isolation of binders specific to the L19-antibody portion of the antigen, we added as competitor 10⁻⁶M L19 antibody in Sip format (WO2003/076469) to the 2% (w/v) skimmed milk/PBS blocking solution (MPBS). L19-IL2-coated dynabeads were incubated with antibody libraries (in 2% MPBS with competitor) for 1 hour at room temperature on hula shaker. Beads were rinsed 6 times with PBS-Tween20 0.1%, and 6 times with PBS. Phage-antibody particles were eluted with 100 mM triethylamine, which was subsequently neutralized by 0.5 mL 1 M Tris-HCl pH 7.4. The eluted phage was used for the infection of exponentially growing E. coli TG1. Two rounds of panning were performed as described.

ELISA

Bacterial supernatants containing scFv fragments were screened for binding to antigen by ELISA (Silacci et al 2005). Individual colonies were inoculated in 180 μl 2TY, 100 μg/ml ampicillin (Applichem; Darmstadt, Germany), 0.1% glucose (Sigma) in 96-well plates (Nunclon Surface, Nunc). The plates were incubated for 3 h at 37° C. in a shaker incubator. The cells were then induced with 1 mM isopropyl-thio-galactopyranoside (IPTG; Applichem), and grown overnight at 30° C. The bacterial supernatants assayed were tested in ELISA experiments as described in Silacci 2005, using the anti-myc tag 9E10 mAb (1 μg/ml) and anti-mouse horseradish peroxidase immunoglobulins (A2554 Sigma) as secondary reagents. The read out was OD450 nm-620 nm to subtract background signal.

Expression and Purification of scFv Antibody Fragments

ScFv antibody fragments from selected bacterial clones were produced by inoculating a single fresh colony in 10 mL 2TY medium, 100 μg/ml ampicillin, and 1% glucose. The pre-culture was grown overnight at 37° C. then diluted 1:100 in 800 ml 2TY medium, 100 μg/ml ampicillin, 0.1% glucose and grown at 37° C. till exponential phase. scFv production was induced by the addition of 1 mM IPTG and grown at 30° C. overnight. The scFv fragments were purified from the bacterial supernatant by affinity chromatography using Protein A Sepharose (Sino Biological Inc.) according to the manufacturer's instructions.

Size-Exclusion Chromatography and BiaCore Analysis

Size-exclusion chromatography was performed on an ÄKTA FPLC system using the Superdex 75 column (Amersham Biosciences). Surface plasmon resonance experiments affinity measurements were performed by BIAcore X100 instrument with purified scFvs. Monomeric scFv were injected as serial-dilution (10⁻⁶ M to 10⁻⁹ M) to accurately measure the KD.

Size-Exclusion Chromatography for L19-IL2/scFv Complex Formation

Size-exclusion chromatography was performed on an ÄKTA FPLC system using the Superdex 200-Increase column (Amersham Biosciences). In order to reach the binding equilibrium, complexes (1:1 molar ratio L19-IL2:scFv) were incubated at room temperature for 1 hour prior to size exclusion analysis.

Sub-Cloning of scFv in Mammalian Expression Vector pCDNA3.1

Antibody fragments were sub-cloned into the mammalian expression vector pCDNA3.1. The nucleotide sequence encoding for a leader peptide required for the secretion of the protein from Chinese Hamster Ovary cells CHO-S

(5′ATGGGCTGGAGCCTGATCCTCCTGTTCCTCGTCGCTGTGGCTACAGGTgtgcacTCG 3′, SEQ ID NO 21) was appended to antibody fragments by PCR, as well as restriction sites HindIII and NotI (at 5′ and 3′, respectively). The restricted DNA was ligated into double digested pCDNA3.1.

Results

Three scFv antibodies named EKH3, PLG5 and PKD7 were isolated. In order to verify the scFv specificity towards IL2, ELISA screening was performed on L19-IL2 biotinylated (target antigen), L19SIP (the competitor used during the selection), and KSF-IL2, another IL2-fusion protein with a different antibody fragment unrelated to L19. The selected clones were positive for both fusion proteins, and were not able to bind to the L19 antibody alone (FIG. 6).

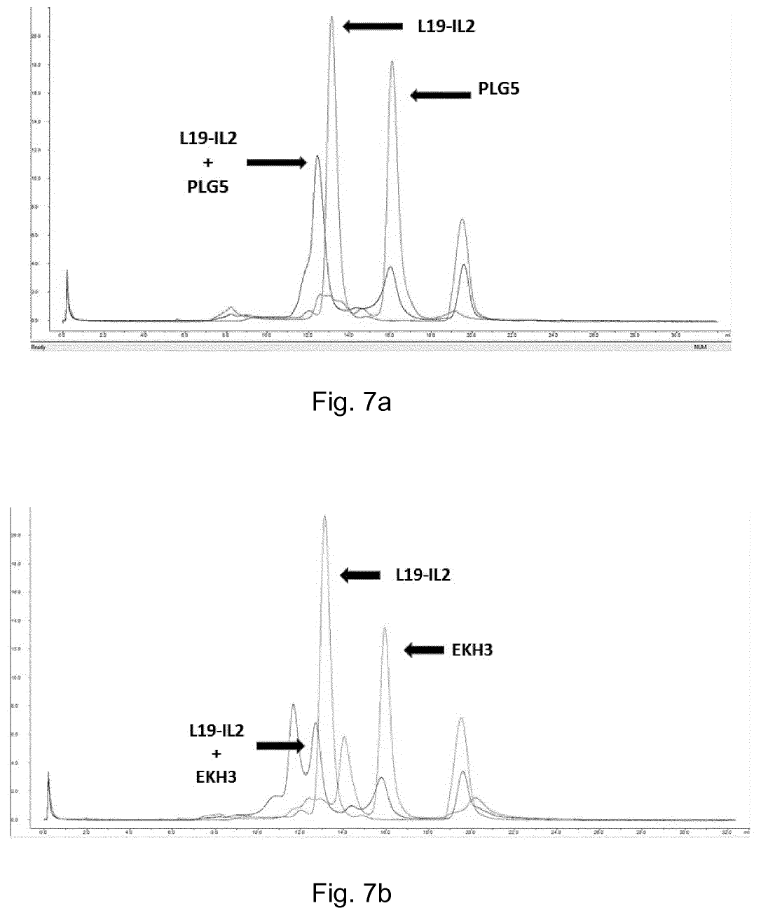

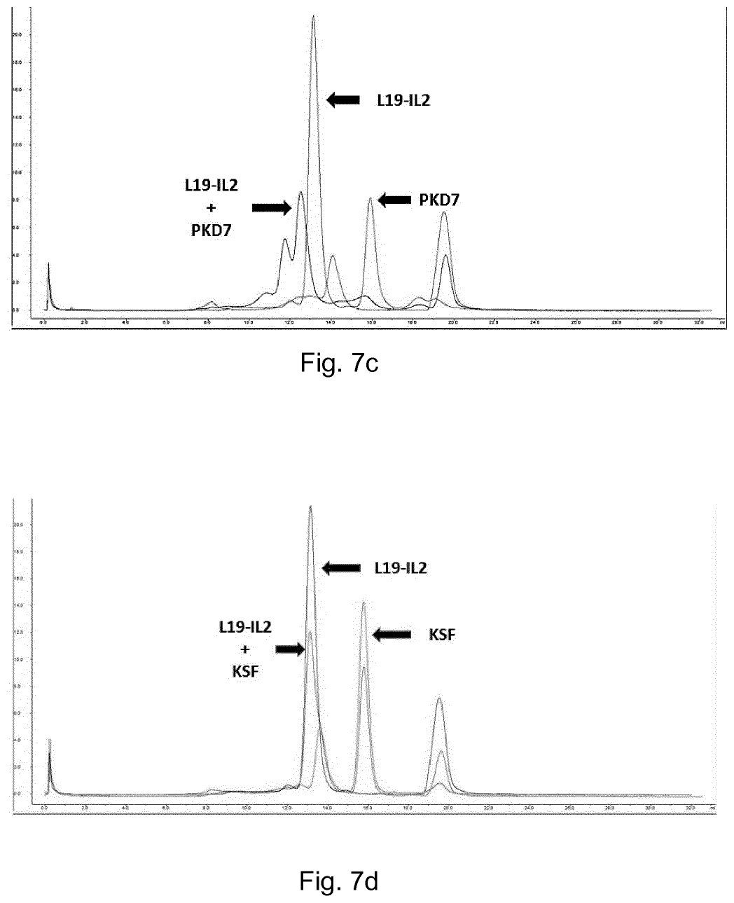

In order to demonstrate that the isolated anti-IL2 scFv were able to form a stable complex in solution with L19-IL2 molecule, size exclusion chromatography under native condition (PBS) was performed on 1:1 complex of L19-IL2:scFv. As described in FIG. 7 (7A to 7C), the binding of the scFv's to the L19-IL2 is able to shift its elution peak to higher molecular weight (corresponding to the sum of the molecular weight of the two proteins), this is not the case for the negative control scFv antibody KSF (7D).

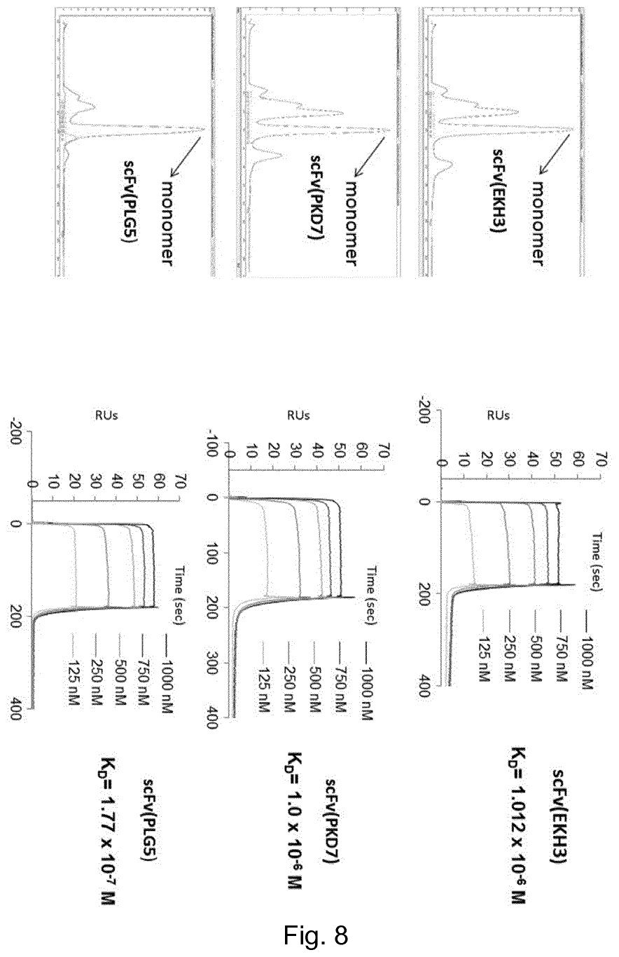

Surface plasmon resonance (SPR) analysis was employed to calculate the affinity constants of the anti-IL2 scFv. Monomeric preparations of the antibody fragment were purified by size exclusion-chromatography (FIG. 8 left panel). SPR was performed on CM5-chip (GE) coated with IL2 cytokine. Biacore analysis demonstrated the specific binding of the new isolated scFv to IL2 alone. Furthermore, it revealed the K_{D} of the anti-IL2 scFv's to be in the low micromolar range (FIG. 8, right panel).

### Example 3: Masking Activity Experiments

Material & Methods

CD1 mice were divided in five groups and received one i.v. injection of the following dosages:

Group 1: 200 μg L19-IL2 (4.8 nmol)

Group 2: 200 μg L19-IL2 (4.8 nmol)+200 μg KSF (8 nmol)

Group 3: 200 μg L19-IL2 (4.8 nmol)+200 μg EKH3 (8 nmol)

Group 4: 200 μg L19-IL2 (4.8 nmol)+160 μg PLG5 (6.4 nmol)

Group 5: 200 μg L19-IL2 (4.8 nmol)+LSD5-61 in 1.25% DMSO (2.5 nmol)

All mice were weighed daily and kept in constant observation for clinical examination. The weight changes of the five groups are reported in FIG. 9.

### FIGURE DESCRIPTION

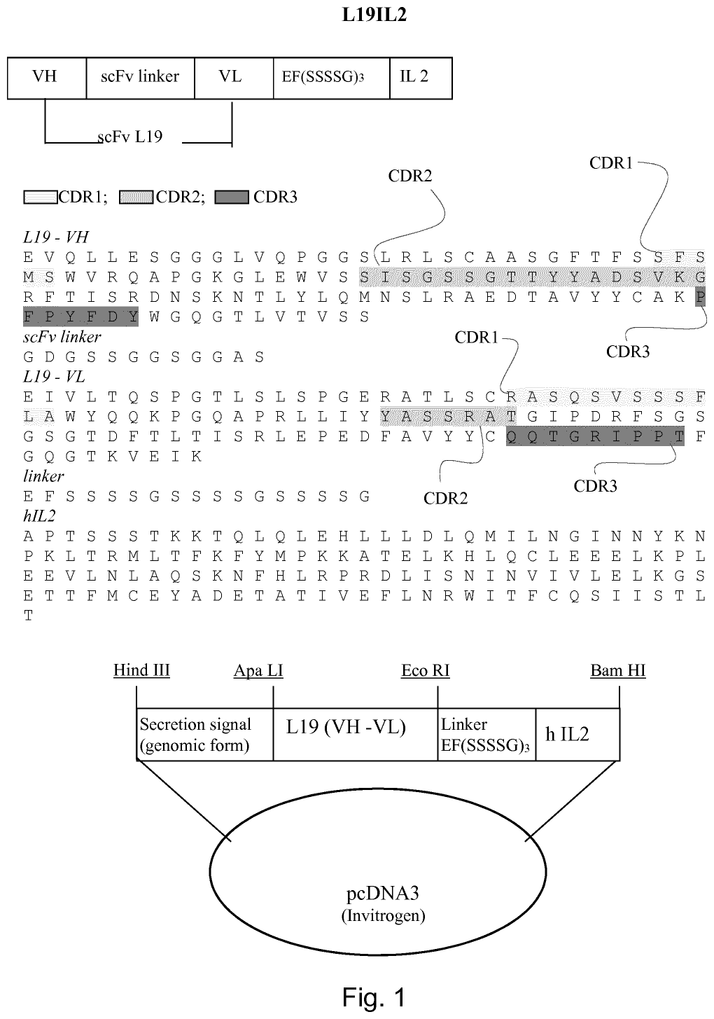

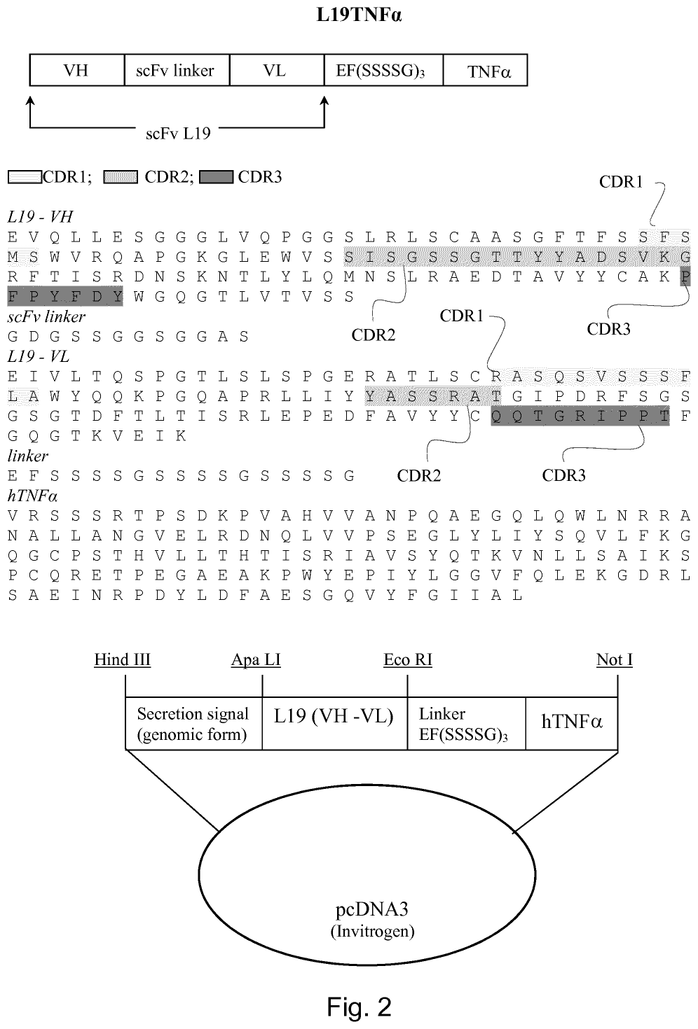

FIGS. 1 and 2 show the sequences and structures of the two immunocytokines L19-IL2 and L19-TNF. The complete sequences thereof are disclosed as SEQ ID No 19 and 20 in the enclosed sequence listing.

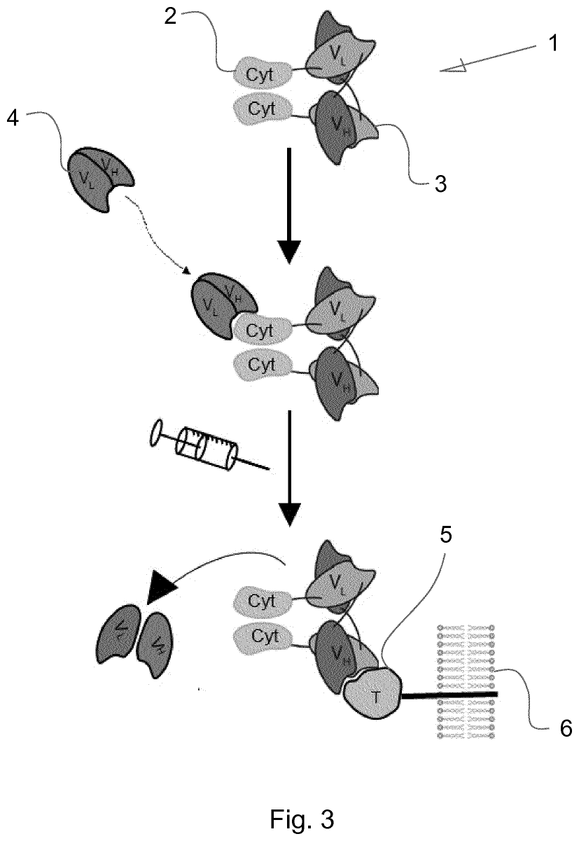

FIG. 3 shows one principle of the invention. An immunocytokine 1 comprising a cytokine 2 and a primary antibody 3 (also called “targeting antibody”) fused or conjugated to one another is mixed with a secondary antibody 4 (also called “masking antibody”), which binds to the cytokine, and inhibits its function. In a more general principle, a primary binding protein or peptide and/or a secondary binding molecule can be used as disclosed in the present specification. After systemic administration to a patient, the targeting antibody of the complex binds to a target structure 5, which is for example present on the neovascolature of tumors.

After binding of the complex, the masking antibody dissociates from the immunocytokine, while the latter remains bound to the target antigen, hence allowing the cytokine to exert its function, e.g., attracting immune cells which then infiltrate the tumor.

As discussed above, one way to achieve this is to modulate the affinities of the primary binding protein (e.g., the targeting antibody) and the secondary binding molecule (e.g., the masking antibody) in such way that the former has a higher affinity to its target than the latter has to the cytokine. More details of this approach are discussed elsewhere herein.

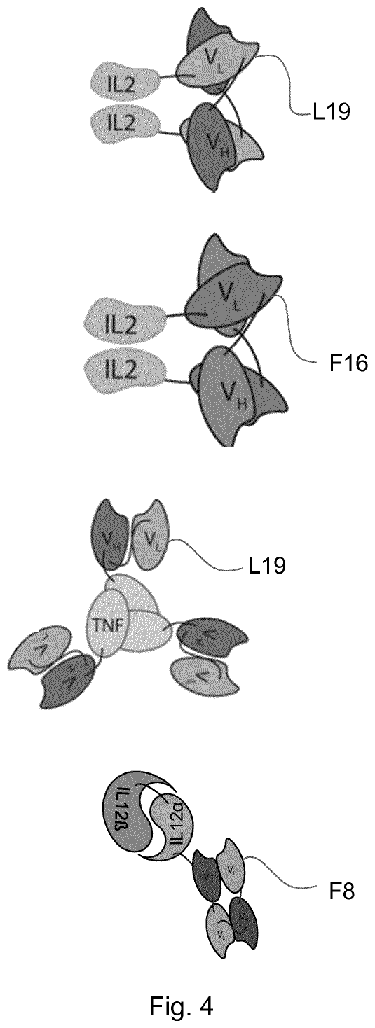

FIG. 4 shows different, non-exhaustive examples of immunocytokines which can be used in the context of the present invention. L19-IL2 is also called Darleukin, F16-IL2 is also called Teleukin, L19-TNF is also called Fibromun. F8-IL12 is also called Dodekin and comprises a single-chain diabody fused to IL12 as disclosed in WO2013/014149.

FIG. 5 shows some details relating to the present invention. FIG. 5A shows the 2 fragments used for the generation of the anti-L19-IL2 binders; FIG. 5B shows a non-exhaustive list of three compounds binding to L19-IL2; FIG. 5C shows the sequencing output of affinity maturation screening; FIG. 5D shows the bifunctional scaffolds bearing a 12-mer DNA tag used for fluorescent polarization analysis, FIG. 5E shows the three compounds binding to L19-IL2 with DNA tag, and FIG. 5F shows the synthesis of LSD5-61.

FIG. 6 shows the results of the ELISA experiments performed on L19-IL2 biotinylated (target antigen), KSF-IL2 (another IL2 fusion protein with an antibody fragment unrelated to the L19), and L19SIP (L19 antibody alone) used as competitor during the selection. The selected antibodies were positive for both fusion proteins, but were not binding to the single L19 antibody alone.

FIG. 7 shows the size exclusion analysis of L19-IL2 complexed with the three different scFv antibodies. FIG. 7A shows the size exclusion analysis of L19-IL2 complexed with PLG5; FIG. 7B shows the size exclusion analysis of L19-IL2 complexed with EKH3; FIG. 7C shows the size exclusion analysis of L19-IL2 complexed with PKD7; and FIG. 7D shows the size exclusion analysis of L19-IL2 complexed with KSF.

FIG. 8 The left panel shows the Size-Exclusion Chomatography for the preparation of the monomeric fraction of each scFv antibody. The right panel shows the sensograms of Surface Plasmon Resonance (Biacore analysis) of each scFv antibody on CM-5 chip coated with Interleukin-2.

FIG. 9 shows the weight changes of the 5 different groups. The group of mice receiving the combination of L19-IL2 and LSD5-61 showed no weight loss as compared to the other four groups. This experiments demonstrate a masking activity of LSD5-61 towards IL2.

Results showing a similar effect of EKH3, PLG5 and other suitable masking antibodies are obtained in corresponding experiments.

### REFERENCES

Bootz and Neri, 2016, Drug Discov Today, 21, 1809
Borsi L. et al (2003) Blood. 102,438492
Borsi L. et al., Int. J. Cancer (2002), 102, 75
Boyman and Sprent, 2012. Nat Rev Immunol, 12, 1800
Brack S. et al. (2006) Clin. Cancer Res., 12, 320008
Carnemolla B. et al., Blood (2002) 99, 165965
Colby D W et al. Methods Enzymol. 2004; 388:348
Curigliano G. et al. Journal of Clinical Oncology (2007) 25, (18S), 3057;
Dandapani S et al., Curr Protoc Chem Biol. 2012; 4: 177-191
Danielli et al., 2015, Cancer Immunol Immunother 64, 99909
De Braud F G. et al (2010) Journal of Clinical Oncology 28 (15S) 13017.
Desnoyer et al. 2016, Sci Transl Med, 5, 207ra144
Frey et al J Urol. 2010 December; 184(6):2540-8
Frey K et al, J Urol 2010; 184:2540-8.
Gafner V et al (2006) Int J Cancer 119: 220512
Gebauer M, Skerra A (2009, Curr Opin Chem Biol. 13 (3): 245-255
Gutbrodt et al. (2013) Sci. Trans. Med., 5, 201ra118
Hemmerle T. et al Br J Cancer. 2013 Sep. 3; 109(5):1206
Hynes, R., (1985) Annu Rev Cell Biol, 1, 67
Köhler G and Milstein C, Nature. Bd. 256, 1975, S. 495-497
Leimbacher M et al. Chemistry. 2012 Jun. 18; 18(25):7729
Lonberg N, Nat Biotechnol. 2005 September; 23(9):1117
Mårlind et al. Clin Cancer Res. 14(20):651524 (2008)
Mazor Y et al., PLoS One. 2016; 11(6)
Moschetta M et al. Cancer Res. 2012 Apr. 1; 72(7):1814
Papadia et al., 2013, J Surg Oncol, 107, 173-179, Schliemann et al., 2015, Cancer Immunol. Res., 3, 547-556 Pasche and Neri, 2012, Drug Discov Today, 17, 5830
Pasche and Neri, 2013, Clin Pharmacol; 5 (Suppl 1): 29-45
Pasche and Neri, 2012, Drug Discov Today, 17, 583-590
Phillips et al., 2008, Anal Chim Acta 621:1018).
Schildbach J F et al. Protein Sci. 1993 February; 2(2):206
Silacci et al., Protein Eng Des Sel. (2006) 19, 471-8.
Sommavilla et al., Protein Eng Des Sel. 2010 August; 23(8):653.
Spitaleri G. et al., (2012) J Cancer Res Clin Oncol. 37, 4475
Villa A, et al Int J Cancer. (2008) 122, 2405.
Wada A, Front Immunol. 2013; 4: 224
Yu Y et al. Science Translational Medicine 25 May 2011, Vol. 3, Issue 84, pp. 84
Zardi et al., Embo J. 1987; 6:233742
Leimbacher et al (2012) Chem. Eur. J. 18, 7729-37
Silacci et al (2005) Proteomics, 5, 2340
WO2010/028791
WO2003/076469
Venetz et al. 2016, J. Biol. Chem, 291, 18139147

Sequences

The sequences shown in the following table are referred to herein. In case there is an ambiguity between this table and the WIPO standard sequence listing that forms part of the present specification and its disclosure, the sequences and qualifiers in this table shall be deemed the correct ones.

| SEQ | Type | Sequence |
|---|---|---|
| 1 | Heavy chain | EVQLLESGGGLVQPGGSLRLSCAASGFTFSSYAMSWVRQAPGKGLEWVSAISGSGGSTYY |
| | variable domain | ADSVKGRFTISRDNSKNTLYLQMNSLRAEDTAVYYCAKRHARYFDYWGQGTLVTVSS |
| | (VH) of EKH3 | |
| 2 | Light chain variable | EIVLTQSPGTLSLSPGERATLSCRASQSVSSSYLAWYQQKPGQAPRLLIYGASSRATGIP |
| | domain (VL) of | DRFSGSGSGTDFTLTISRLEPEDFAVYYCQQTWGSPPTFGQGTKVEIK |
| | EKH3 | |
| 3 | EKH3 scFv | EVQLLESGGGLVQPGGSLRLSCAASGFTFSSYAMSWVRQAPGKGLEWVSAISGSGGSTYY |
| | | ADSVKGRFTISRDNSKNTLYLQMNSLRAEDTAVYYCAKRHARYFDYWGQGTLVTVSSGGG |
| | | GSGGGGSGGGGEIVLTQSPGTLSLSPGERATLSCRASQSVSSSYLAWYQQKPGQAPRLLI |
| | | YGASSRATGIPDRFSGSGSGTDFTLTISRLEPEDFAVYYCQQTWGSPPTFGQGTKVEIK |
| 4 | HCDR1 of EKH3 | SSYAMS |
| 5 | HCDR2 of EKH3 | AISGSGGSTYYADSVKG |
| 6 | HCDR3 of EKH3 | RHARYFDY |
| 7 | LCDR1 of EKH3 | RASQSVSSSYLA |
| 8 | LCDR2 of EKH3 | GASSRAT |
| 9 | LCDR3 of EKH3 | QQTWGSPPT |
| 10 | Heavy chain | EVQLLESGGGLVQPGGSLRLSCAASGFTFSSYAMSWVRQAPGKGLEWVSAINGSGGSTYY |
| | variable domain | ADSVKGRFTISRDNSKNTLYLQMNSLRAEDTAVYYCAKRRGPSFDYWGQGTLVTVSS |
| | (VH) of PLG5 | |
| 11 | Light chain variable | EIVLTQSPGTLSLSPGERATLSCRASQSVSSSYLAWYQQKPGQAPRLLIYGASSRATGIP |
| | domain (VL) of | DRFSGSGSGTDFTLTISRLEPEDFAVYYCQQGPGGPATFGQGTKVEIK |
| | PLG5 | |
| 12 | PLG5 scFv | EVQLLESGGGLVQPGGSLRLSCAASGFTFSSYAMSWVRQAPGKGLEWVSAINGSGGSTYY |
| | | ADSVKGRFTISRDNSKNTLYLQMNSLRAEDTAVYYCAKRRGPSFDYWGQGTLVTVSSGGG |
| | | GSGGGGSGGGGEIVLTQSPGTLSLSPGERATLSCRASQSVSSSYLAWYQQKPGQAPRLLI |
| | | YGASSRATGIPDRFSGSGSGTDFTLTISRLEPEDFAVYYCQQGPGGPATFGQGTKVEIK |
| 13 | HCDR1 of PLG5 | SSYAMS |
| 14 | HCDR2 of PLG5 | AINGSGGSTYYADSVKG |
| 15 | HCDR3 of PLG5 | RRGPSFDY |
| 16 | LCDR1 of PLG5 | RASQSVSSSYLA |
| 17 | LCDR2 of PLG5 | GASSRAT |
| 18 | LCDR3 of PLG5 | QQGPGGPAT |
| 19 | L19-IL2 | EVQLLESGGGLVQPGGSLRLSCAASGFTFSSFSMSWVRQAPGKGLEWVSSISGSSGTTYY |
| | | ADSVKGRFTISRDNSKNTLYLQMNSLRAEDTAVYYCAKPFPYFDYWGQGTLVTVSSGDGS |
| | | SGGSGGASEIVLTQSPGTLSLSPGERATLSCRASQSVSSSFLAWYQQKPGQAPRLLIYYA |
| | | SSRATGIPDRFSGSGSGTDFTLTISRLEPEDFAVYYCQQTGRIPPTFGQGTKVEIKEFSS |
| | | SSGSSSSGSSSSGAPTSSSTKKTQLQLEHLLLDLQMILNGINNYKNPKLTRMLTFKFYMP |
| | | KKATELKHLQCLEEELKPLEEVLNLAQSKNFHLRPRDLISNINVIVLELKGSETTFMCEY |
| | | ADETATIVEFLNRWITFCQSIISTLT |
| 20 | L19-TNF | EVQLLESGGGLVQPGGSLRLSCAASGFTFSSFSMSWVRQAPGKGLEWVSSISGSSGTTYY |
| | | ADSVKGRFTISRDNSKNTLYLQMNSLRAEDTAVYYCAKPFPYFDYWGQGTLVTVSSGDGS |
| | | SGGSGGASTQSPGTLSLSPGERATLSCRASQSVSSSFLAWYQQKPGQAPRLLIYYASSRA |
| | | TGIPDRFSGSGSGTDFTLTISRLEPEDFAVYYCQQTGRIPPTFGQGTKVEIKEFSSSSGS |
| | | SSSGSSSSGVRSSSRTPSDKPVAHVVANPQAEGQLQWLNRRANALLANGVELRDNQLVVP |
| | | SEGLYLIYSQVLFKGQGCPSTHVLLTHTISRIAVSYQTKVNLLSAIKSPCQRETPEGAEA |
| | | KPWYEPIYLGGVFQLEKGDRLSAEINRPDYLDFAESGQVYFGIIAL |
| 21 | leader peptide for | ATGGGCTGGAGCCTGATCCTCCTGTTCCTCGTCGCTGTGGCTACAGGTGTGCACTCG |
| | Chinese Hamster | |
| | Ovary cells CHO-S | |

1. A combination comprising at least
a) an immunocytokine comprising at least
a1) a primary binding protein and
a2) a cytokine
fused or conjugated to one another,
wherein the primary binding protein is the anti-EDB antibody L19; and
b) a secondary binding molecule capable of binding to at least a section of at least one cytokine comprised in the immunocytokine,
wherein the secondary binding molecule is selected from the group consisting of
a monoclonal antibody, or a fragment thereof, or
a small molecule.

2.-7. (canceled)

8. The combination according to claim 1, wherein the monoclonal antibody or fragment thereof comprises
a heavy chain variable domain VH having the sequence set forth in SEQ ID NO: 1, and a light chain variable domain VL having the sequence set forth in SEQ ID NO: 2, or
a heavy chain variable domain VH having the sequence set forth in SEQ ID NO: 10, and a light chain variable domain VL having the sequence set forth in SEQ ID NO: 11.

9. The combination according to claim 1, wherein the monoclonal antibody or fragment thereof adopts a format selected from the group consisting of:
IgG
a Fab fragment, a F(ab′)2 fragment,
a Fv (variant fragment), a scFv (single-chain variant fragment) or a scFv-Fc and/or
a domain antibody (dAb) fragment, or a diabody.

10. (canceled)

11. (canceled)

12. The combination according to claim 1, wherein the secondary binding molecule
a) is conjugated to an entity that reduces cell membrane permeation,
b) comprises a moiety that binds to an entity that reduces cell membrane permeation, and/or
c) has a given polarity or charge.

13. The combination according to claim 1, wherein the cytokine is an inflammatory cytokine.

14. The combination according to claim 1, wherein at least one secondary binding molecule is smaller than, or equally sized as, the primary binding protein or peptide.

15. The combination according to claim 1, wherein at least one secondary binding molecule has an affinity towards the cytokine which is essentially equal as, or similar to, the affinity the respective receptor has to the cytokine.

16. The combination according to claim 1, wherein at least one secondary binding molecule has an affinity towards the cytokine which is smaller than, or equal as, the affinity the primary binding protein or peptide has to its target.

17. A complex comprising the combination according to claim 1, in which complex at least one secondary binding molecule is bound to at least one cytokine comprised in the immunocytokine.

18. A pharmaceutical composition comprising the combination according to claim 1, plus at least one further pharmaceutically acceptable ingredient.

19. A method of preparing the combination according to claim 1, comprising the steps of:
a) providing the immunocytokine
b) providing the secondary binding molecule, and
c) mixing the two.

20. A method of treating a human or animal subject suffering from, at risk of developing and/or being diagnosed for a disease that is indicated for treatment with an immunocytokine, or for the prevention of such condition, comprising administering the combination of claim 1 to the subject.

21. The method according to claim 20, wherein the pathologic condition is a neoplastic disease.

22. The combination according to claim 1, wherein
the small molecule is LSD5-61, and/or
the monoclonal antibody, or fragment thereof, comprises
(i) a VH domain comprising a framework and a set of complementarity determining regions HCDR1, HCDR2 and HCDR3, and
(ii) a VL domain comprising a framework and a set of complementarity determining regions LCDR1, LCDR2 and LCDR3
wherein the set of 6 CDRs is selected from the following:
SEQ ID NOs 4-9, or
SEQ ID Nos 13-18.


SEQUENCE LISTING (21 sequences)

SEQ ID NO: 1 - protein, 117 aa, Artificial Sequence
  Heavy chain variable domain (VH) of EKH3
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRHARYFDYWGQGTLVTVSS

SEQ ID NO: 2 - protein, 108 aa, Artificial Sequence
  Light chain variable domain (VL) of EKH3
EIVLTQSPGTLSLSPGERATLSCRASQSVSSSYLAWYQQKPGQAPRLLIYGASSRATGIPDRFSGSGSGTDFTLTISRLEPEDFAVYYCQQTWGSPPTFGQGTKVEIK

SEQ ID NO: 3 - protein, 239 aa, Artificial Sequence
  EKH3 scFv
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKRHARYFDYWGQGTLVTVSSGGGGSGGGGSGGGGEIVLTQSPGTLSLSPGERATLSCRASQSVSSSYLAWYQQKPGQAPRLLIYGASSRATGIPDRFSGSGSGTDFTLTISRLEPEDFAVYYCQQTWGSPPTFGQGTKVEIK

SEQ ID NO: 4 - protein, 6 aa, Artificial Sequence
  CDR1 of heavy chain of EKH3
SSYAMS

SEQ ID NO: 5 - protein, 17 aa, Artificial Sequence
  CDR2 of heavy chain of EKH3
AISGSGGSTYYADSVKG

SEQ ID NO: 6 - protein, 8 aa, Artificial Sequence
  CDR3 of heavy chain of EKH3
RHARYFDY

SEQ ID NO: 7 - protein, 12 aa, Artificial Sequence
  CDR1 of light chain of EKH3
RASQSVSSSYLA

SEQ ID NO: 8 - protein, 7 aa, Artificial Sequence
  CDR2 of light chain of EKH3
GASSRAT

SEQ ID NO: 9 - protein, 9 aa, Artificial Sequence
  CDR3 of light chain of EKH3
QQTWGSPPT

SEQ ID NO: 10 - protein, 117 aa, Artificial Sequence
  Heavy chain variable domain (VH) of PLG5
EVQLLESGGGLVQPGGSLRLSCAASGFTFSSYAMSWVRQAPGKGLEWVSAINGSGGSTYYADSVKGRFTISRDNSKNTLYLQMNSLRAEDTAVYYCAKRRGPSFDYWGQGTLVTVSS

SEQ ID NO: 11 - protein, 108 aa, Artificial Sequence
  Light chain variable domain (VL) of PLG5
EIVLTQSPGTLSLSPGERATLSCRASQSVSSSYLAWYQQKPGQAPRLLIYGASSRATGIPDRFSGSGSGTDFTLTISRLEPEDFAVYYCQQGPGGPATFGQGTKVEIK

SEQ ID NO: 12 - protein, 239 aa, Artificial Sequence
  PLG5 scFv
EVQLLESGGGLVQPGGSLRLSCAASGFTFSSYAMSWVRQAPGKGLEWVSAINGSGGSTYYADSVKGRFTISRDNSKNTLYLQMNSLRAEDTAVYYCAKRRGPSFDYWGQGTLVTVSSGGGGSGGGGSGGGGEIVLTQSPGTLSLSPGERATLSCRASQSVSSSYLAWYQQKPGQAPRLLIYGASSRATGIPDRFSGSGSGTDFTLTISRLEPEDFAVYYCQQGPGGPATFGQGTKVEIK

SEQ ID NO: 13 - protein, 6 aa, Artificial Sequence
  CDR1 of heavy chain of PLG5
SSYAMS

SEQ ID NO: 14 - protein, 17 aa, Artificial Sequence
  CDR2 of heavy chain of PLG5
AINGSGGSTYYADSVKG

SEQ ID NO: 15 - protein, 8 aa, Artificial Sequence
  CDR3 of heavy chain of PLG5
RRGPSFDY

SEQ ID NO: 16 - protein, 12 aa, Artificial Sequence
  CDR1 of light chain of PLG5
RASQSVSSSYLA

SEQ ID NO: 17 - protein, 7 aa, Artificial Sequence
  CDR2 of light chain of PLG5
GASSRAT

SEQ ID NO: 18 - protein, 9 aa, Artificial Sequence
  CDR3 of light chain of PLG5
QQGPGGPAT

SEQ ID NO: 19 - protein, 386 aa, Artificial Sequence
  L19-IL2
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKEFSSSSGSSSSGSSSSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 20 - protein, 406 aa, Artificial Sequence
  L19-TNF
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKEFSSSSGSSSSGSSSSGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 21 - DNA, 57 bp, Artificial Sequence
  leader peptide for Chinese Hamster Ovary cells CHO-S
atgggctggagcctgatcctcctgttcctcgtcgctgtggctacaggtgtgcactcg